(19) United States
(12) Patent Application Publication
(10) Pub. No.: US 2021/0163532 A1
(43) Pub. Date: Jun. 3, 2021

(54) NOVEL MACROCYCLIC OPIOID PEPTIDES

(71) Applicant: University of Florida Research Foundation, Incorporated, Gainesville, FL (US)

(72) Inventors: Jane V. Aldrich, Gainesville, FL (US); Michael J. Ferracane, Irvine, CA (US)

(73) Assignee: University of Florida Research Foundation, Incorporated, Gainesville, FL (US)

(21) Appl. No.: 17/047,977

(22) PCT Filed: Apr. 17, 2019
(86) PCT No.: PCT/US2019/027928

§ 371 Date: Oct. 15, 2020

Related U.S. Application Data
(60) Provisional application No. 62/658,915, filed on Apr. 17, 2018.

(51) Int. Cl. C07K 5/12; C07K 7/64; A61P 25/04

(52) U.S. Cl. CPC C07K 5/126; A61K 38/00; A61P 25/04; C07K 7/64

(57) ABSTRACT

The invention relates to macrocyclic peptides and pharmaceutical compositions thereof. The invention further provides macrocyclic tetrapeptides comprising sarcosine. The invention further relates to pharmaceutical compositions for modulating opioid receptor activity. The macrocyclic tetrapeptides provided herein are useful in treating diseases or disorders relating to the activity of one or more opioid receptors, such as neurological disorders.

### RELATED APPLICATIONS

This application claims the benefit under 35 U.S.C. § 119(e) of the filing date of U.S. Provisional Application Ser. No. 62/658,915, filed Apr. 17, 2018, entitled “NOVEL MACROCYCLIC OPIOID PEPTIDES”, the entire contents of which is incorporated by reference herein.

### STATEMENT OF GOVERNMENT SUPPORT

This invention was made with government support under Grant No. DA018832 awarded by the National Institutes of Health; and Grant Nos. W81XWH-15-1-0452 and W81XWH-15-1-0464 awarded by U.S. Army Medical Research and Medical Materiel Command. The government has certain rights in the invention.

### BACKGROUND

Opioid receptors belong to the Type A class of G-protein coupled receptors (GPCRs) and bind to opioid ligands. The three major types of opioid receptors are the delta (δ) opioid receptor (DOR), kappa (κ) opioid receptor (KOR), and mu (μ) opioid receptor (MOR). The nociceptin receptor (NOR) was later identified as a fourth major opioid receptor type, as it shares >60% sequence homology with the δ-, κ- and μ-receptors. The delta, kappa, and mu opioid receptors exhibit approximately 50% sequence similarity [Reisine, T. and Bell, G. I. (1993) Molecular biology of opioid receptors Trends Neurosci 16, 506-510]. Opioid receptors fulfill a variety of functions within the cell, including activation of ion channels, inhibition of neurotransmitter release, and inhibition of adenylyl cyclase to decrease intracellular levels of cAMP. The distinct anatomical distributions of each receptor contribute to their mediation of different behaviors [Corbett, A. D., Henderson, G., McKnight, A. T., and Paterson, S. J. (2006) 75 years of opioid research: the exciting but vain quest for the Holy Grail. Br J Pharmacol 147, S153-S162]. Opioid receptors are widely distributed throughout the body and have distinct endogenous ligands.

The delta (δ) opioid receptor (DOR) has enkephalins as its endogenous ligand. The delta opioid receptor is found in the brain (e.g., in the pontine nuclei, amygdala, olfactory bulbs, and deep cortex) and in peripheral sensory neurons. Delta opioid receptors are distributed in brain regions associated with processes involved in the perception of pain, sensory information, emotional processing, and impulsivity, among others, indicating that DOR agonists and antagonists could be effective at treating a variety of indications, such as depression and other mood disorders, along with providing analgesic effects [Peppin, J. F. and Raffa, R. B. (2015) Delta opioid agonists: a concise update on potential therapeutic applications. J Clin Pharm Ther 40, 155-166]. While the exact role of the DOR in pain modulation is debated, it has been suggested that the DOR modulates the nociception of chronic pain [Berrocoso, E., Sánchez-Blázquez, P., Garzón, J., and Mico, J. A. (2009) Opiates as antidepressants. Curr Pharm Des 15, 1612-1622].

The mu (μ) opioid receptor (MOR) binds enkaphalins and beta-endorphin as endogenous ligands with high affinity. The mu opioid receptor is found in the brain (e.g., cortex, thalamus, striosomes, periaqueductal gray, and rostral ventromedial medulla), spinal cord (e.g., substantia gelatinosa), peripheral sensory neurons, and intestinal tract. Morphine was the agonist used to originally define MOR. Long-term or high-dose use of opioids can lead to the development of tolerance, including downregulation of MOR gene expression or the upregulation of glutamate pathways in the brain that exert an opioid-opposing effect to reduce the effect of opioids [Ueda, H. and Ueda, M. (2009) Mechanisms underlying morphine analgesic tolerance and dependence. Front Biosci 14, 5260-5272]. Agonists of the MOR have long been used to treat pain, but are limited by their side effects [Stein, C. (2016) Annu Rev Med 67, 433-451].

The kappa (κ) opioid receptor (KOR) binds the opioid peptides the dynorphins as the primary endogenous ligands. The kappa opioid receptor is found in the brain (e.g., hypothalamus, periaqueductal gray, and claustrum), spinal cord (e.g., substantial gelatinosa), and peripheral sensory neurons. KOR agonists are involved in pain modulation, hallucinogenic or dissociative effects, and chronic stress (e.g., depression, anxiety, anhedonia, and increased drug-seeking behavior). KOR agonists have been investigated for their potential in the treatment of addiction [Hasebe, K., Kawai, K., Suzuki, T., Kawamura, K., Tanaka, T., Narita, M., Nagase, H., and Suzuki, T. (2004) Possible pharmacotherapy of the opioid kappa receptor agonist for drug dependence. Ann NY Acad Sci 1025, 404-413]. However, KOR has also been shown to influence stress-induced relapse to drug seeking behavior, where the longer effects of KOR agonism have been linked to KOR-dependent stress-induced potentiation of reward behavior and reinstatement of drug seeking [Beardsley, P. M., Howard, J. L., Shelton, K. L., and Carroll, F. I. (2005) Differential effects of the novel kappa opioid receptor antagonist, JDTic, on reinstatement of cocaine-seeking induced by footshock stressors vs cocaine primes and its antidepressant-like effects in rats. Psychopharmacology (Berl) 183, 118-126; Redila, V. A., and Chavkin, C. (2008) Stress-induced reinstatement of cocaine seeking is mediated by the kappa opioid system Psychopharmacology (Berl) 200, 59-70].

Addiction to drugs, including cocaine and alcohol, continues to be a world-wide issue. Despite sustained efforts to develop methods for prevention and/or treatment of addiction, there is an unmet need for improvement of current therapies directed toward this goal. There is a continued need to develop therapeutics that selectively target opioid receptors, are orally available, and readily cross the blood-brain barrier to penetrate the central nervous system (CNS). Notably, pretreatment with KOR antagonists can prevent stress-induced reinstatement of cocaine-seeking behavior, as well as decrease compulsive cocaine-intake in the absence of stress [Carey, A. N., Borozny, K., Aldrich, J. V., and McLaughlin, J. P., (2007) Reinstatement of cocaine place-conditioning prevented by the peptide kappa-opioid receptor antagonist, Arodyn Eur J Pharmacol 569, 84-89; Ross, N. C., Reilley, K. J., Murray, T. F., Aldrich, J. V., and McLaughlin, J. P. (2012) Novel opioid cyclic tetrapeptides: Trp isomers of CJ-15,208 exhibit distinct opioid receptor agonism and short-acting κ opioid receptor antagonism Br J Pharmacol 165, 1097-1108; Wee, S. Orio, L., Ghirmai, S., Cashman, J. R., and Koob, G. F. (2009) Inhibition of kappa opioid receptors attenuated increased cocaine intake in rats with extended access to cocaine Psychopharmacology (Berl) 205, 565-575; Wee, S., Vendruscolo, L. F., Misra, K. K., Scholsburg, J. E., and Koob, G. F. (2012) A combination of buprenorphine and naltrexone blocks compulsive cocaine intake in rodents without producing dependence Sci Transl Med 4, 146ra110]. Several non-peptide KOR antagonists (e.g., nor-binaltorphimine, 5-guanidinylnaltrindole, and JDTic), however, exhibit unusually long duration of antagonism despite having the desired high selectivity for KOR, limiting their clinical development [Metcalf, M. D., and Coop, A. (2005) Kappa opioid antagonists: past successes and future prospects AAPS J 7, E704-722; Horan, P, Taylor, J., Yamamura, H. I., and Porreca, F. (1991) Extremely long-lasting antagonistic actions of nor-binaltorphimine (nor-BNI) in the mouse tail-flick test J Pharmacol Exp Ther 260, 1237-1243; Carroll, I., Thomas, J. B., Dykstra, L. A., Granger, A. L., Allen, R. M., Howard, J. L., Pollard, G. T., Aceto, M. D., and Harris, L. S. (2004) Pharmacological properties of JDTic: a novel kappa-opioid receptor antagonist Eur J Pharmacol 501, 111-119]. These studies suggest that molecules with KOR activity, both KOR agonists and antagonists, hold promise as medications to prevent addiction relapse and treat other CNS-related disorders (e.g., depression, anxiety, mood disorders, convulsions, and nociception).

### BRIEF SUMMARY OF THE INVENTION

The natural product macrocyclic tetrapeptide CJ-15,208 (cyclo[Phe-D-Pro-Phe-Trp]) and its D-Trp isomer have been shown to bind to and selectively antagonize KOR in vitro [Ross, N. C., Kulkarni, S. S., McLaughlin, J. P., and Aldrich, J. V. (2010) Synthesis of CJ-15,208, a novel κ-opioid receptor antagonist Tetrahedron Lett 51, 5020-5023; Ross et al., Br. J. Pharmacol. 2012; U.S. Pat. No. 8,809,278; WO 2016/007956]. Additionally, both peptides demonstrate opioid activity in vivo and have the ability to prevent the reinstatement of previously extinguished cocaine seeking behavior [Ross et al., Br. J. Pharmacol. 2012; Aldrich, J. V., Senadheera, S. N., Ross, N. C., Ganno, M. L., Eans, S. O., and McLaughlin, J. P. (2013) The macrocyclic peptide CJ-15,208 is orally active and prevents reinstatement of extinguished cocaine-seeking behavior J Nat Prod 76, 433-438; Eans, S. O., Ganno, M. L., Reilley, K. J., Patkar, K. A., Senadheera, S. N., Aldrich, J. V. and McLaughlin, J. P. (2013) The macrocyclic tetrapeptide [D-Trp]CJ-15,208 produces short acting κ opioid receptor antagonism in the CNS after oral administration Br J Pharmacol 169, 426-436]. The invention is based, at least in part, on the improvement of in vivo opioid activity of novel macrocyclic tetrapeptides. Herein we describe the development and characterization of macrocyclic tetrapeptides comprising one or more N-methyl residues, for example, cyclo[D-Phe-Pro-Sar-Phe] (Sar=sarcosine, N-methylglycine). Importantly, in addition to exhibiting improved metabolic stability in vitro, these novel macrocyclic tetrapeptides potently and selectively antagonize the kappa opioid receptor (KOR) in vivo.

Thus, in some aspects, the invention is directed toward macrocyclic compounds and methods of synthesis, their mechanism of action, methods of modulating opioid receptor activity, and methods of treating disease and disorders associated with the target of the macrocyclic compounds. In some aspects, provided herein are compounds for use in treating one or more diseases or disorders associated with the function of an opioid receptor.

In one aspect, the invention provides a compound of Formula (1), or a solvate or hydrate thereof:

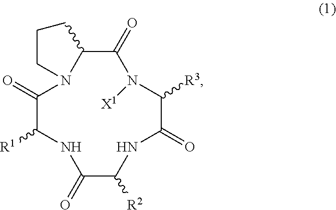

wherein each R¹, R², and R³ is independently hydrogen or an amino acid side chain; and X¹ is a substituted or unsubstituted alkyl. In certain embodiments, the amino acid side chain is the side chain of a naturally occurring amino acid. In certain embodiments, the amino acid side chain is the side chain of an unnatural amino acid. In certain embodiments, the amino acid side chain is a phenylalanine side chain. In certain embodiments, the amino acid side chain is a tryptophan side chain. In certain embodiments, R³ is hydrogen. In certain embodiments, X¹ is substituted or unsubstituted C₁₋₆ alkyl. In some embodiments, X¹ is methyl.

In another aspect, the invention provides a compound of Formula (1), wherein the compound is:

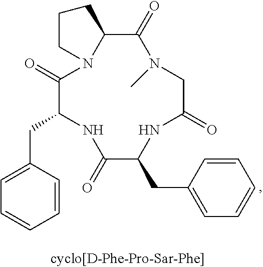 [SMILES: CN1CC(=O)N[C@@H](Cc2ccccc2)C(=O)N[C@H](Cc2ccccc2)C(=O)N2CCC[C@H]2C1=O]

or a solvate or hydrate thereof.

In certain embodiments, a compound of Formula (1) modulates one or more opioid receptors. In certain embodiments, the compound activates one or more opioid receptors. In certain embodiments, the compound blocks the activation of one or more opioid receptors.

In a further aspect, provided herein are compounds of Formula (1), wherein the compound is a opioid receptor antagonist. In certain embodiments, the compound is a kappa opioid receptor antagonist.

In yet another aspect, provided herein are compounds of Formula (1), wherein the compound is a opioid receptor agonist. In certain embodiments, the compound is a mu opioid receptor agonist. In some embodiments, the compound is a mu opioid receptor agonist and a kappa opioid receptor agonist.

Also provided herein are pharmaceutical compositions and kits comprising a compound of Formula (1). In certain embodiments, the pharmaceutical composition comprises a pharmaceutically acceptable adjuvant, carrier, or excipient. In certain embodiments, the pharmaceutical composition further comprises one or more additional therapeutic agents. In some embodiments, the kit provides one or more doses of a compound or pharmaceutical composition described herein for treating a subject with a disease or disorder described herein.

In some aspects, provided herein are methods for treating a disease, disorder, or symptom thereof in a subject, the method comprising administering to the subject a compound, or a pharmaceutical composition thereof, of any of the formulae provided herein. In certain embodiments, the subject is a human.

In a further aspect, the invention provides method of treating a disease, disorder, or symptom thereof in a subject, the method comprising administering to the subject a compound of any of the formulae provided herein in combination with one or more additional therapeutic agents, or a pharmaceutical composition thereof.

In another aspect, provided herein are methods for treating a subject with a neurological disorder, psychiatric disorder, painful condition, or opioid receptor mediated disorder, or symptoms thereof, comprising administering to the subject in need thereof, an effective amount of a compound, or pharmaceutical composition thereof, of any of the formulae provided herein. In some embodiments, the subject is suffering from an addiction.

In another aspect, provided herein are methods for treating a subject in need of an analgesic. In certain embodiments, the method comprises administering to the subject in need thereof, an effective amount of a compound, or pharmaceutical composition thereof, of any of the formulae provided herein.

The details of one or more embodiments of the invention are set forth in the accompanying Figures, the Detailed Description, and the Examples. Other features, objects, and advantages of the invention will be apparent from the description and the claims.

### BRIEF DESCRIPTION OF THE DRAWINGS

The present invention is further described below with reference to the following non-limiting examples and with reference to the following figures, in which:

FIG. 1 shows dose- and time-dependent antinociception and antagonism of the KOR agonist U50,488 by cyclo[D-Phe-Pro-Sar-Phe] (JVA-4001) after i.c.v. administration.

FIGS. 2A to 2B. FIG. 2A shows opioid receptor selectivity of KOR antagonism produced by 0.01 nmol i.c.v. cyclo[D-Phe-Pro-Sar-Phe] (JVA4001). FIG. 2B shows the duration of antagonism produced by 0.01 nmol i.c.v. cyclo[D-Phe-Pro-Sar-Phe] (JVA4001).

FIG. 3 shows dose- and time-dependent antinociception, but not antagonism of the KOR agonist U50,488, produced by cyclo[D-Phe-Pro-D-NMe-Ala-Phe] (JVA-4002) administered i.c.v.

FIG. 4 shows opioid receptor involvement in cyclo[D-Phe-Pro-Sar-Phe]-induced antinociception in the 55° C. warm-water tail-withdrawal assay after i.c.v. administration using MOR KO, KOR KO, or C57BL/6J mice pretreated with naltrindole (0.5 mg/kg, s.c., −20 min) in the 55° C. warm-water tail-withdrawal assay. A subset of KOR KO mice received β-FNA (5 mg/kg, s.c., −24 h; pink square) prior to testing of the cyclic tetrapeptide. Tail-withdrawal latencies were measured 20 min after cyclo[D-Phe-Pro-Sar-Phe] administration. Points represent average % antinociception ±SEM from 5-8 mice for each set is presented.

FIGS. 5A and 5B show characterization of opioid receptor antagonist activity of cyclo[D-Phe-Pro-Sar-Phe] in the mouse 55° C. warm-water tail-withdrawal assay. FIG. 5A—Mice pretreated 2.5 h with cyclo[D-Phe-Pro-Sar-Phe] demonstrate dose-dependent antagonism of U50,488-induced antinociception after i.p administration. The antinociceptive effect of U50,488 (10 mg/kg, i.p.) was determined in mice pretreated for 2.5 or 4.5 h with cyclo[D-Phe-Pro-Sar-Phe] (0.01 nmol, i.c.v). Mean % antinociception SEM from 8-18 mice is presented. * significantly different from response of agonist alone. FIG. 5B shows the antinociceptive activity of cyclo[Pro-Sar-Phe-D-Phe] following i.p. dosing in the 55° C. warm-water tail-withdrawal assay in C57Bl/6J mice. cyclo[Pro-Sar-Phe-D-Phe]demonstrated significant time- and dose-dependent antinociception with repeated measurement over time. Points represent average % antinociception ±SEM from 8-24 mice for each set presented.

FIGS. 6A to 6C. FIG. 6A shows locomotor activity of cyclo[D-Phe-Pro-Sar-Phe] after i.p. administration to C57BL/6J mice in the rotarod assay. Mice received i.p. cyclo[D-Phe-Pro-Sar-Phe] (10 mg/kg) U50,488 (10 mg/kg), vehicle (10% Solutol in 0.9% saline), or 0.9% saline, and were tested on the rotarod apparatus by repeated measurements over time. Latencies to fall are given as the mean % change from baseline (100%) performance ±SEM. Data from 8-16 mice is presented. * significantly different from response of saline alone. FIGS. 6B and C show effects of cyclo[D-Phe-Pro-Sar-Phe] on ambulation (FIG. 6B) and respiration (FIG. 6C) in C57BL76J mice tested in the CLAMS/Oxymax system. Ambulation and respiration were monitored after i.p. administration of cyclo[D-Phe-Pro-Sar-Phe] (10 mg/kg, n=10), or morphine (10 mg/kg, n=16). Data presentenced as % vehicle response ±SEM; ambulation, XAMB (A) or breaths per minute, BPM (B). *=significantly different from baseline response (p<0.05). †=p<0.05 vs morphine.

FIG. 7 shows the daily conditioning of C57BL/6J mice for 2 days with 0.9% saline (i.c.v), morphine (30 nmol, i.c.v.), U50,488 (100 nmol, i.c.v), or cyclo[D-Phe-Pro-Sar-Phe](0.1-10 nmol, i.c.v.) with a counterbalanced design. Data is presented as mean difference in time spent on the drug-paired side ±SEM (n=16-28). * significantly different from matching preconditioning preference.

FIGS. 8A to 8B. FIG. 8A shows the preference of mice for the morphine-paired environment following 4 days of morphine administration (10 mg·kg¹, i.p. daily), with extinction occurring 8 weeks later. Mice were then exposed to forced swim stress or another round of morphine place conditioning, reinstating preference. Pretreatment with the cyclo[D-Phe-Pro-Sar-Phe] (0.01 nmol, i.c.v.) prevented stress-induced reinstatement of place preference, and pretreatment with the cyclo[D-Phe-Pro-Sar-Phe] (0.03 nmol, i.c.v.) prevented morphine-induced reinstatement of place preference. Further, a round of conditioning with cyclo[D-Phe-Pro-Sar-Phe] (0.01 nmol, i.c.v.) did not in itself cause reinstatement of place preference. FIG. 8B shows that peripheral pretreatment with cyclo[D-Phe-Pro-Sar-Phe] or nor-BNI (each at 10 mg/kg, i.p.) prevented stress-induced reinstatement of place preference. Bars represent means of n=12-46 mice (B) or n=14-21 mice.

FIG. 9 shows dose-dependent antinociception and antagonism of the KOR agonist U50,488 (administered peripherally (i.p.) or centrally (i.c.v.)) by cyclo[D-Phe-Pro-Sar-Phe](JVA 4001) following per os (p.o.) administration.

FIGS. 10A to B. FIG. 10A shows that cyclo[D-Phe-Pro-Sar-Phe] (JVA 4001, 0.01 nmol, i.c.v.) prevented both stress- and cocaine-induced reinstatement of cocaine CPP. *, p<0.05 compared to preconditioning response; p<0.05 compared to t post-place conditioning and ‡ matching reinstatement condition. FIG. 10B shows that cyclo[D-Phe-Pro-Sar-Phe](JVA 4001) prevented both stress- and cocaine-induced reinstatement of CPP after oral administration. *, p<0.05 compared to preconditioning response; p<0.05 compared to † post-place conditioning and ‡ matching reinstatement condition.

FIG. 11 shows that cyclo[D-Phe-Pro-Sar-Phe] (JVA 4001, 0.01 nmol, i.c.v.) prevented both stress- and ethanol-induced reinstatement of ethanol CPP. *, p<0.05 compared to preconditioning response; p<0.05 compared to † post-place conditioning and ‡ matching reinstatement condition.

### DEFINITIONS

Before further description of the present invention, and in order that the invention may be more readily understood, certain terms are first defined and collected here for convenience.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

The term “isomers” or “stereoisomers” refers to compounds which have identical chemical constitution, but differ with regard to the arrangement of the atoms or groups in space.

The term “enantiomers” refers to two stereoisomers of a compound which are non-superimposable mirror images of one another. An equimolar mixture of two enantiomers is called a “racemic mixture” or a “racemate.”

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

With respect to the nomenclature of a chiral center, terms “D” and “L” with respect to configuration are as defined by the IUPAC Recommendations. As to the use of the terms, diastereomer, racemate, epimer and enantiomer will be used in their normal context to describe the stereochemistry of preparations.

In a formula, [symbol] is a single bond where the stereochemistry of the moieties immediately attached thereto is not specified but can be any stereochemistry (i.e., D or L).

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn). In certain embodiments, the alkyl group is an unsubstituted C₁₋₆ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu))). In certain embodiments, the alkyl group is a substituted C₁₋₆ alkyl (e.g., —CF₃, Bn).

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “amino acid” refers to naturally occurring and synthetic amino acids, as well as amino acid analogs and amino acid mimetics that function in a manner similar to the naturally occurring amino acids. Every amino acid contains an amine (—NH₂) and a carboxylic acid (—COOH) functional group. Each amino acid contains a unique side chain, designated by the “R” substituent shown below.

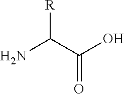

Naturally occurring amino acids are those encoded by the genetic code, as well as those amino acids that are later modified, e.g., hydroxyproline, γ-carboxyglutamate, and O-phosphoserine. In certain embodiments, the amino acid is an N-alkyl amino acid, where the hydrogen on any non-proline amine (N) is replaced with an alkyl (e.g., methyl (—CH₃)) group. In certain embodiments, the N-alkyl amino acid is sarcosine (Sar). Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Unnatural (or non-natural) amino acids refer to those not naturally incorporated into proteins during translation. Examples of unnatural amino acids include, but are not limited to, β-amino acids (e.g., β² and β³), homo-amino acids, proline derivatives, pyruvic acid derivatives, 3-substituted alanine derivatives, alanine derivatives (e.g., 1′- and 2′-naphthylalanine) glycine derivatives, ring-substituted phenylalanine and tyrosine derivatives, linear core amino acids, and N-methyl amino acids. Amino acids may be referred to herein by either their commonly known three letter symbols or by the one-letter symbols recommended by the IUPAC-IUB Biochemical Nomenclature Commission. As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. In certain embodiments, a compound of Formula (1) provided herein comprises an amino acid side chain selected from the 20 proteinogenic amino acids (i.e., an amino acid incorporated into proteins during translation) shown in Table 1. The term amino acid may also refer to non-proteinogenic amino acids, such as, for example, selenocysteine (—CH₂SeH).

TABLE 1
Isoproteinogenic amino acids and side chains.

| | | | Side chain |
|---|---|---|---|
| | 3-letter | | classification |
| Amino Acid | code | Side chain (R group) | (at pH 7.4) |
| Arginine | Arg | —(CH₂)₃NHC(NH)NH₂ | Charged |
| Histidine | His | —CH₂—C₃H₃N₂ | (Positive) |
| Lysine | Lys | —(CH₂)₄NH₂ | |
| Aspartic acid | Asp | —CH₂COOH | Charged |
| (aspartate) | | | (Negative) |
| Glutamic acid | Glu | —CH₂CH₂COOH | |
| (glutamate) | | | |
| Serine | Ser | —CH₂OH | Uncharged |
| Threonine | Thr | —CH(OH)CH₃ | |
| Asparagine | Asn | —CH₂CONH₂ | |
| Glutamine | Gln | —CH₂CH₂CONH₂ | |
| Cysteine | Cys | —CH₂SH | Special |
| Glycine | Gly | —H | |
| Proline | Pro | —CH₂CH₂CH₂— | Hydrophobic |
| Alanine | Ala | —CH₃ | |
| Isoleucine | Ile | —CH(CH₃)CH₂CH₃ | |
| Leucine | Leu | —CH₂CH(CH₃)₂ | |
| Methionine | Met | —CH₂CH₂SCH₃ | |
| Phenylalanine | Phe | —CH₂C₆H₅ | |
| Tryptophan | Trp | —CH₂C₈H₆N | |
| Tyrosine | Tyr | —CH₂—C₆H₄OH | |
| Valine | Val | —CH(CH₃)₂ | |

A “peptide” is a sequence of at least two amino acids. Peptides can consist of short as well as long amino acid sequences, including proteins. Peptides can be derived naturally or synthetically. Peptides can contain natural and non-natural amino acids, e.g. synthetic amino acids. A “macrocyclic peptide” is a cyclized peptide. In some embodiments, a macrocyclic peptide comprises a ring comprising about 12 or more atoms. In some embodiments, a macrocylic peptide comprises a ring comprising between 3 and 50 amino acid residues. In some embodiments, a macrocylic peptide comprises a ring comprising between 3 and 20 amino acid residues. In some embodiments, a macrocylic peptide comprises a ring comprising between 3 and 10 amino acid residues. In some embodiments, a macrocylic peptide comprises a ring comprising between 3 and 9 amino acid residues. In some embodiments, a macrocylic peptide comprises a ring comprising between 3 and 8 amino acid residues. In some embodiments, a macrocylic peptide comprises a ring comprising between 3 and 7 amino acid residues. In some embodiments, a macrocylic peptide comprises a ring comprising between 3 and 6 amino acid residues. In some embodiments, a macrocylic peptide comprises a ring comprising between 3 and 5 amino acid residues. In some embodiments, a macrocylic peptide comprises a ring comprising 4 amino acid residues. The term “protein” refers to series of amino acid residues connected one to the other by peptide bonds between the alpha-amino and carboxy groups of adjacent residues. Without wishing to be bound by any particular theory, a protein generally comprises about 50 or more amino acid residues.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. Typical domains are made up of sections of organization such as stretches of β-sheet and α-helices. “Tertiary structure” refers to the complete three dimensional structure of a polypeptide monomer and can contain different domains which are portions of a polypeptide that form a compact unit of the polypeptide and are typically 50 to 350 amino acids long. “Quaternary structure” refers to the three dimensional structure formed by the noncovalent association of independent tertiary units.

The term “receptor” refers to any protein molecule that receives a signal from outside or inside a cell. In general, receptors are membrane bound proteins. A receptor may be a peripheral membrane protein, an integral membrane protein, or any protein that interacts with the cellular membrane. A receptor may be comprised of a single protein or a complex of two or more proteins. Receptors induce a type of cellular response when a chemical signal or molecule binds to the receptor. A receptor may also refer to any drug target, such as an enzyme, transporter, or ion channel that is the target of a drug. In general, any molecule that binds to or interacts with a receptor is referred to as a ligand. Examples of receptors include, but are not limited to, ionotropic receptors, G-protein coupled receptors, receptor tyrosine kinases, and nuclear receptors.

The term “opioid receptor” refers to any inhibitory G protein-coupled receptor with an opioid as a ligand. The opioid receptor may be located pre-synaptically or post-synaptically. The opioid receptors referred to herein may be any of the major types of opioid receptors, including but not limited to the delta (δ) opioid receptor (DOR, OP₁), kappa (κ) opioid receptor (KOR, OP₂), mu (μ) opioid receptor (MOR, OP₃), and nociceptin receptor (NOR, OP₄). The term “opioid receptor” further encompasses any homomeric or heteromeric combination of the opioid receptors described above. The opioid receptors described herein include opioid receptors derived from any source or any tissue or cell type.

The term “delta opioid receptor” refers to the delta-1 opioid receptor (δ₁) and delta-2 opioid receptor (δ₂) and any combination or variation thereof. In certain aspects, delta opioid receptors are preferred. A delta opioid receptor can be located anywhere in the body, including the brain (e.g., pontine nucleus, amygdala, olfactory bulbs, and deep cortex). A delta opioid receptor mediates a variety of responses, including analgesia, euphoria, antidepressant effects, convulsant effects, and physical dependence.

The term “kappa opioid receptor” refers to the kappa-1 opioid receptor (κ₁), kappa-2 opioid receptor (κ₂), kappa-3 opioid receptor (κ₃) and any combination or variation thereof. In certain aspects, kappa opioid receptors are preferred. A kappa opioid receptor can be located anywhere in the body, including the brain (e.g., hypothalamus, periaqueductal gray, and claustrum) and spinal cord (e.g., substantia gelatinosa). A kappa opioid receptor mediates a variety of responses, including spinal analgesia, anticonvulsant effects, depression, dissociative effects, hallucinogenic effects, dysphoria, neuroprotection, stress, sedation, miosis, physical dependence, and diuresis.

The term “mu opioid receptor” refers to the mu-1 opioid receptor (μ₁), mu-2 opioid receptor (μ₂), mu-3 opioid receptor (μ₃) and any combination or variation thereof. In certain aspects, mu opioid receptors are preferred. A mu opioid receptor can be located anywhere in the body, including the brain (e.g., laminae III of the cortex, laminae IV of the cortex, thalamus, and periaqueductal gray) and spinal cord (e.g., substantia gelatinosa). A mu opioid receptor mediates a variety of responses, including supraspinal analgesia, physical dependence, respiratory depression, miosis, euphoria, vasodilation, and reduced gastrointestinal motility.

The term “nociceptin opioid receptor” refers to the nociceptin-1 opioid receptor (ORL₁) and any combination or variation thereof. A nociceptin opioid receptor can be located anywhere in the body, including brain (e.g., cortex, amygdala, hippocampus, septal nuclei, habenula, and hypothalamus) and the spinal cord. A nociceptin opioid receptor mediates a variety of responses, including anxiety, depression, appetite, and development of tolerance to mu-opioid agonists.

The term “ligand” refers to any molecule of any composition that interacts with or binds to a biomolecule, protein, or receptor. Ligands typically form a complex with a biomolecule, protein, or receptor to serve a biological purpose. Binding of a ligand often results in a change in conformation of the target biomolecule. A ligand can be a small molecule, a peptide, an ion, a protein, an amino acid, a polymer, a nucleotide, a nucleic acid, DNA, RNA, or any derivatives thereof. The term “ligand” encompasses agonists, partial agonists, mixed agonist-antagonists, antagonists, inverse agonists, and allosteric modulators, among others. In certain embodiments, the ligand is a macrocyclic peptide comprising between 3 to 20 amino acids. In certain embodiments, the ligand is a macrocyclic tetrapeptide (i.e., 4 amino acids). In certain embodiments, the ligand is a compound of Formula (1). In certain embodiments, the ligand is cyclo(D-Phe-Pro-Sar-Phe) (also called JVA-4001 throughout).

The term “agonist” refers to any chemical or molecule that binds either reversibly or irreversibly to a receptor and activates said receptor to produce a biological response. An agonist further refers to any chemical or molecule that causes an action or outcome (e.g., within a cell) as a result of binding to or interacting with a receptor. An agonist can be an endogenous agonist that is naturally produced by the body (e.g., a hormone or neurotransmitter) or an exogenous agonist (e.g., a drug). An agonist further encompasses all types of agonists, including superagonists, full agonists, partial agonists, silent agonists, partial inverse agonists, full inverse agonists, co-agonists, and irreversible agonists. In certain embodiments, the agonist is a peptide comprising between 1-20 amino acids, inclusive. In certain embodiments, the agonist is a macrocyclic peptide comprising between 2-20 amino acids, inclusive. In certain embodiments, the agonist is a macrocyclic peptide comprising between 3-20 amino acids, inclusive. In certain embodiments, the agonist is a macrocyclic peptide comprising between 3-15 amino acids, inclusive. In certain embodiments, the agonist is a macrocyclic peptide comprising between 3-10 amino acids, inclusive. In certain embodiments, the agonist is a macrocyclic peptide comprising between 3-9 amino acids, inclusive. In certain embodiments, the agonist is a macrocyclic peptide comprising between 3-8 amino acids, inclusive. In certain embodiments, the agonist is a macrocyclic peptide comprising between 3-7 amino acids, inclusive. In certain embodiments, the agonist is a macrocyclic peptide comprising between 3-6 amino acids, inclusive. In certain embodiments, the agonist is a macrocyclic peptide comprising between 3-5 amino acids, inclusive. In certain embodiments, the agonist is a macrocyclic tetrapeptide (i.e. 4 amino acids). In certain embodiments, the agonist is a compound of Formula (1). In certain embodiments, the agonist is cyclo(D-Phe-Pro-Sar-Phe).

The term “antagonist” refers to any chemical or molecule that binds either reversibly or irreversibly to a receptor and blocks or reduces a biological response from said receptor. An antagonist further refers to any chemical or molecule that blocks or reduces an action or outcome (e.g., within a cell) as a result of binding to or interacting with a receptor. Antagonists may be referred to as blockers (e.g., alpha blockers, beta blockers, calcium channel blockers, etc.). An antagonist is any chemical or molecule that has affinity but no efficacy for a receptor. An antagonist can block the action of an agonist. An antagonist can bind to any location of the receptor (e.g., to an active site, an allosteric site, or any binding site not involved in the regulation of a receptor's activity). An antagonist can be an endogenous ligand that is naturally produced by the body (e.g., a hormone or neurotransmitter) or an exogenous antagonist (e.g., a drug). In certain embodiments, the antagonist is a peptide comprising between 1-20 amino acids, inclusive. In certain embodiments, the antagonist is a macrocyclic peptide comprising between 2-20 amino acids, inclusive. In certain embodiments, the antagonist is a macrocyclic peptide comprising between 3-20 amino acids, inclusive. In certain embodiments, the antagonist is a macrocyclic peptide comprising between 3-15 amino acids, inclusive. In certain embodiments, the antagonist is a macrocyclic peptide comprising between 3-10 amino acids, inclusive. In certain embodiments, the antagonist is a macrocyclic peptide comprising between 3-9 amino acids, inclusive. In certain embodiments, the antagonist is a macrocyclic peptide comprising between 3-8 amino acids, inclusive. In certain embodiments, the antagonist is a macrocyclic peptide comprising between 3-7 amino acids, inclusive. In certain embodiments, the antagonist is a macrocyclic peptide comprising between 3-6 amino acids, inclusive. In certain embodiments, the antagonist is a macrocyclic peptide comprising between 3-5 amino acids, inclusive. In certain embodiments, the antagonist is a macrocyclic tetrapeptide (i.e. 4 amino acids). In certain embodiments, the antagonist is a compound of Formula (1). In certain embodiments, the antagonist is cyclo(D-Phe-Pro-Sar-Phe).

The term “mixed agonist/antagonist” refers to any chemical or molecule that has the properties and/or functions of both an agonist and an antagonist. A mixed agonist/antagonist can be an endogenous mixed agonist/antagonist that is naturally produced by the body (e.g., a hormone or neurotransmitter) or an exogenous mixed agonist/antagonist (e.g., a drug). For example, without wishing to be bound by any particular theory, a compound of the invention may display agonist activity upon administration to a subject, and then antagonistic activity be apparent after a period of time following administration (i.e., sustained antagonistic activity). In some embodiments, the compound is an agonist of one or more opioid receptors upon administration to a subject, and an antagonist of one or more opioid receptors after a period of time (e.g., 1 hour, 2 hours, 3 hours, 4 hours, 5 hours, 6 hours, 7 hours, 8 hours, 9 hours, 10 hours, 11 hours, 12 hours, 1 day, etc.) following administration. In some embodiments, the compound is a kappa opioid receptor mixed agonist/antagonist. In some embodiments, the compound is a mu opioid receptor agonist upon administration to a subject, and a mu opioid receptor antagonist after a period of time following administration. In certain embodiments, the mixed agonist/antagonist is a peptide comprising between 1-20 amino acids, inclusive. In certain embodiments, the mixed agonist/antagonist is a macrocyclic peptide comprising between 2-20 amino acids, inclusive. In certain embodiments, the mixed agonist/antagonist is a macrocyclic peptide comprising between 3-20 amino acids, inclusive. In certain embodiments, the mixed agonist/antagonist is a macrocyclic peptide comprising between 3-15 amino acids, inclusive. In certain embodiments, the mixed agonist/antagonist is a macrocyclic peptide comprising between 3-10 amino acids, inclusive. In certain embodiments, the mixed agonist/antagonist is a macrocyclic peptide comprising between 3-9 amino acids, inclusive. In certain embodiments, the mixed agonist/antagonist is a macrocyclic peptide comprising between 3-8 amino acids, inclusive. In certain embodiments, the mixed agonist/antagonist is a macrocyclic peptide comprising between 3-7 amino acids, inclusive. In certain embodiments, the mixed agonist/antagonist is a macrocyclic peptide comprising between 3-6 amino acids, inclusive. In certain embodiments, the mixed agonist/antagonist is a macrocyclic peptide comprising between 3-5 amino acids, inclusive. In certain embodiments, the mixed agonist/antagonist is a macrocyclic tetrapeptide (i.e. 4 amino acids). In certain embodiments, the mixed agonist/antagonist is a macrocyclic tetrapeptide (i.e. 4 amino acids). In certain embodiments, the mixed agonist/antagonist is a compound of Formula (1). In certain embodiments, the agonist is cyclo(D-Phe-Pro-Sar-Phe).

The phrase “opioid activity” refers to any activity that is associated with an opioid receptor. Opioid activity may refer to a molecule acting as an agonist, an antagonist, or mixed agonist/antagonist.

The phrase “acting as an antagonist” refers to any molecule, ligand, or compound that performs the function of an “antagonist,” as defined above.

The phrase “acting as an agonist” refers to any molecule, ligand, or compound that performs the function of an “agonist,” as defined above.

The phrase “acting as a mixed agonist/antagonist” refers to any molecule, ligand, or compound that performs the function of a “mixed agonist/antagonist,” as defined above.

The terms “agent” is used herein to refer to any substance, compound (e.g., molecule), supramolecular complex, material, or combination or mixture thereof. A compound may be any agent that can be represented by a chemical formula, chemical structure, or sequence. Examples of agents, include, e.g., small molecules, polypeptides, nucleic acids, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Embodiments exhibiting alternative protonation states, configurations (e.g., geometric or stereoisomeric forms), solvates, and forms are encompassed by the present disclosure where applicable. In certain embodiments, the agent is a therapeutic compound (e.g., small molecule, peptide, macrocyclic peptide, etc.) that is useful for treating a subject with a neurological disorder, psychiatric disorder, painful condition, or opioid receptor mediated disorder, or symptoms thereof.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal, intracerebroventricularly), oral, pulmonary (e.g., inhalation), rectal, or transdermal administration. The pharmaceutical preparations may be given by forms suitable for each administration route. For example, these preparations are administered in tablets or capsule form, by injection, infusion, or inhalation; topically by lotion or ointment; and rectally by suppositories. In certain embodiments, the composition is administered enterally. In certain embodiments, the pharmaceutical composition is formulated for oral administration. In certain embodiments, the composition is administered parenterally. In certain embodiments, the pharmaceutical composition is formulated for intravenous administration. In certain embodiments, the injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in combination with either another agent as described above or with a pharmaceutically acceptable carrier, or both. The compound of the invention can be administered prior to the administration of the other agent, simultaneously with the agent, or after the administration of the agent.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, an effective amount is an amount sufficient to reduce symptoms associated with a neurological disorder, psychiatric disorder, painful condition, or opioid receptor mediated disorder. In certain embodiments, an effective amount is an amount sufficient to reduce symptoms associated with an addiction. In certain embodiments, an effective amount is an amount sufficient to treat a subject with a neurological disorder, psychiatric disorder, painful condition, or opioid receptor mediated disorder. In certain embodiments, an effective amount is an amount sufficient to treat an addiction.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, the therapeutically effective amount refers to an amount of an agent which is effective, upon single or multiple dose administration to the patient, in reducing and/or alleviating the symptoms of an opioid receptor mediated disorder, or in prolonging the survivability of the patient with such an opioid receptor mediated disorder beyond that expected in the absence of such treatment. The term “therapeutically effective amount” can encompass an amount that improves overall therapy, reduces or avoids symptoms, signs, or causes of the condition, and/or enhances the therapeutic efficacy of another therapeutic agent. In certain embodiments, the therapeutically effective amount is an amount sufficient for modulating an opioid receptor. In certain embodiments, a therapeutically effective amount is an amount sufficient for blocking the activity of an opioid receptor. In certain embodiments, the therapeutically effective amount is an amount sufficient for activating an opioid receptor. In certain embodiments, a therapeutically effective amount is an amount sufficient for treating a drug or alcohol addiction. In certain embodiments, a therapeutically effective amount is an amount sufficient for treating drug or alcohol abuse. In certain embodiments, a therapeutically effective amount is an amount sufficient for treating a morphine addiction. In certain embodiments, a therapeutically effective amount is an amount sufficient for treating cocaine addiction. In certain embodiments, a therapeutically effective amount is an amount sufficient for treating a stress-induced disorder. In certain embodiments, a therapeutically effective amount is an amount sufficient for treating a chronic relapsing disorder. In certain embodiments, a therapeutically effective amount is an amount sufficient for treating or reducing drug-seeking behavior. In certain embodiments, a therapeutically effective amount is an amount sufficient for treating or reducing stress-induced drug-seeking behavior. In certain embodiments, a therapeutically effective amount is an amount sufficient for treating stress-induced reinstatement of cocaine-seeking behavior. In certain embodiments, a therapeutically effective amount is an amount sufficient for treating stress-induced reinstatement of morphine-seeking behavior. In certain embodiments, the therapeutically effective amount is an amount sufficient for treating stress-induced reinstatement of morphine-seeking behavior. In certain embodiments, a therapeutically effective amount is an amount sufficient for treating a drug or alcohol addiction as a result of increased opioid receptor activity. In certain embodiments, a therapeutically effective amount is an amount sufficient for treating a drug or alcohol addiction as a result of reduced opioid receptor activity. In certain embodiments, a therapeutically effective amount is an amount sufficient for treating stress-induced reinstatement of cocaine-seeking behavior as a result of reduced opioid receptor activity. In certain embodiments, the therapeutically effective amount is an amount sufficient for treating a disease resulting, at least in part, from reduced kappa opioid receptor (KOR) activity. In certain embodiments, the therapeutically effective amount is an amount sufficient for treating a disease resulting, at least in part, from reduced mu opioid receptor (MOR) activity.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “modulate” refers to altering the function or activity (e.g., a biological response) of a protein. An agent (e.g., an agonist) may modulate a protein by making it more active or inducing a function. An agent may modulate a protein by reducing its activity or inhibiting a function. For example, an antagonist can interact with a protein and interfere with the normal binding of the agonist, thereby blocking the protein (e.g., a receptor protein) and preventing a biological response.

The term “subject” includes organisms which are capable of suffering from a disease or disorder or who could otherwise benefit from the administration of a compound of the invention. In certain embodiments, the subject is a human or a non-human animal. In certain embodiments, the subject is a human with a neurological disorder, psychiatric disorder, painful condition, or opioid receptor mediated disorder, or symptoms thereof. The term “non-human animals” of the invention includes all vertebrates, e.g., mammals; e.g., rodents (e.g., mice); non-human primates; sheep, dog, cow, etc. and non-mammals, such as chickens, amphibians, reptiles, etc.

The terms “condition,” “disease,” and “disorder” are used interchangeably.

The phrase “stress-induced reinstatement of cocaine-seeking behavior” refers to cocaine-seeking behavior that is promoted by stress. In general, stress increases the endogenous levels of dynorphin (Dyn). Dyn is the endogenous ligand of the kappa opioid receptor (KOR).

The phrase “stress-induced reinstatement of morphine-seeking behavior” refers to morphine-seeking behavior that is promoted by stress.

The phrase “drug-induced reinstatement of drug-seeking behavior” refers to drug-seeking behavior that is promoted by exposure to a small dose of the drug.

The phrase “cocaine-induced reinstatement of cocaine-seeking behavior” refers to cocaine-seeking behavior that is promoted by exposure to a small dose of cocaine.

The phrase “morphine-induced reinstatement of morphine-seeking behavior” refers to morphine-seeking behavior that is promoted by exposure to a small dose of morphine.

The phrase “opioid receptor mediated disorder” refers to any disease or disorder caused by upregulation (e.g., increased function) or downregulation (e.g., decreased function) of opioid receptor function. An opioid receptor mediated disorder includes, in some embodiments, neurological disorders and psychiatric disorders. An opioid receptor mediated disorder includes stress-induced reinstatement of drug-seeking behavior and drug-induced reinstatement of drug-seeking behavior. In certain embodiments, the stress-induced reinstatement of drug-seeking behavior is stress-induced reinstatement of cocaine-seeking behavior. In certain embodiments, the stress-induced reinstatement of drug-seeking behavior is stress-induced reinstatement of ethanol-seeking behavior. In certain embodiments, the stress-induced reinstatement of drug-seeking behavior is stress-induced reinstatement of opioid-seeking behavior. In certain embodiments, the drug-induced reinstatement of drug-seeking behavior is cocaine-induced reinstatement of cocaine-seeking behavior. In certain embodiments, the drug-induced reinstatement of drug-seeking behavior is ethanol-induced reinstatement of ethanol-seeking behavior. In certain embodiments, the drug-induced reinstatement of drug-seeking behavior is opioid-induced reinstatement of opioid-seeking behavior.

The term “reinstatement” and “relapse” can be used interchangeably (e.g., are synonyms).

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, including, but not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome. In certain embodiments, a neurological disease that is addiction is preferred.

A “painful condition” includes, but is not limited to, neuropathic pain (e.g., peripheral neuropathic pain), central pain, deafferentation pain, chronic pain (e.g., chronic nociceptive pain, and other forms of chronic pain), post-operative pain (e.g., pain arising after hip, knee, or other replacement surgery), pre-operative pain, stimulus of nociceptive receptors (nociceptive pain), acute pain (e.g., phantom and transient acute pain), fibromyalgia, noninflammatory pain, inflammatory pain, pain associated with cancer, wound pain, burn pain, acute post-operative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition. In certain embodiments, the painful condition is nociceptive pain. In certain embodiments, the painful condition is pain associated with withdrawal symptoms from drug addiction.

In certain embodiments, the painful condition is nociceptive pain. The term “nociceptive pain” refers to pain resulting from stimulation of nociceptive receptors. Without wishing to be bound by any particular theory, nociceptive pain may be caused by a chemical (e.g., capsaicin), mechanical (e.g., cutting), or thermal (e.g., hot or cold) stimulus. Nociceptive pain also includes visceral pain (i.e., pain that results from the activation of nociceptors Nociceptive pain also includes pain resulting from the alteration of nociception as a result of alteration of opioid receptor function.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia). In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulation of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

In certain embodiments, the painful condition is inflammatory pain. In certain embodiments, the painful condition (e.g., inflammatory pain) is associated with an inflammatory condition and/or an immune disorder.

The term “addiction” refers to a disease of the mind characterized by compulsive engagement in rewarding or addictive stimuli. An addiction often involves addictive stimuli that are reinforcing (e.g., increase the likelihood that a person will seek repeated exposure to the agent causing the stimulus) and intrinsically rewarding (e.g., they are perceived by a person as being inherently desirable, positive, and pleasurable). The addiction may arise through transcriptional or epigenetic mechanisms and generally develops over time as a result of persistent exposure to addictive stimulus or stimuli. Cognitive control, particularly inhibitory control over behavior, is impaired in a person suffering from addiction. Additionally, stimulus-driven behavioral responses (i.e., stimulus control) that are associated with a particular rewarding stimulus tend to dominate the behavior of a person suffering from addiction. The term addiction encompasses addiction to drugs (e.g., cocaine, morphine, opioids, and the like), alcohol, gambling, etc. In certain embodiments, the addiction is a drug addiction. In certain embodiments, the addiction is a cocaine addiction. In certain embodiments, the addiction is an ethanol addiction. In certain embodiments, the addiction is an opioid addiction.

The term “psychiatric disorder” refers to a disease of the mind and includes diseases and disorders listed in the Diagnostic and Statistical Manual of Mental Disorders—Fifth Edition (DSM-IV), published by the American Psychiatric Association, Washington D.C. (2013). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, Cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

### DETAILED DESCRIPTION OF THE INVENTION

As generally described herein, the present invention is based on the derivatization of macrocyclic peptides that possess in vivo opioid activity. These macrocyclic peptides possess higher in vitro stability. In some aspects, these novel macrocyclic tetrapeptides potently and selectively antagonize an opioid receptor in vivo.

In some aspects, the invention provides macrocyclic tetrapeptides which may be represented by Formula (1):

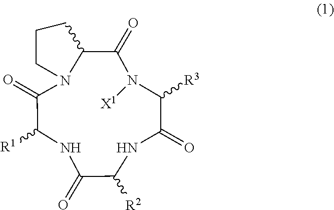

or a solvate or hydrate thereof;
wherein

each R¹, R², and R³ is independently hydrogen or an amino acid side chain; and

X¹ is substituted or unsubstituted alkyl.

As referred to throughout the present disclosure, an amino acid side chain may be chosen from any side chain shown in Table 1 (“R group”), or a derivative thereof. Alternatively, an amino acid side chain may be an unnatural amino acid, as described herein, or a derivative thereof. In some embodiments, the unnatural amino acid is 1′-naphthylalanine. In some embodiments, the unnatural amino acid is 2′-naphthylalanine.

In certain embodiments, R¹ is a phenylalanine side chain. In certain embodiments, R¹ is hydrogen.

In certain embodiments, R² is a phenylalanine side chain. In certain embodiments, R² is a tryptophan side chain. In certain embodiments, R² is an alanine side chain. In certain embodiments, R² is hydrogen. In certain embodiments, R² is not hydrogen.

In certain embodiments, R³ is hydrogen. In certain embodiments, R³ is an alanine side chain.

In certain embodiments, X¹ is a substituted or unsubstituted C₁₋₆ alkyl. In certain embodiments, X¹ is a substituted or unsubstituted C₂₋₆ alkyl. In certain embodiments, X¹ is methyl. In certain embodiments, X¹ is ethyl.

In certain embodiments, R¹ and R² are phenylalanine side chains. In certain embodiments, R¹ and R² are phenylalanine side chains, and R³ is hydrogen. In certain embodiments, R¹ is a phenylalanine side chain and R² is a tryptophan side chain. In certain embodiments, R¹ is a phenylalanine side chain, R² is a tryptophan side chain, and R³ is hydrogen.

In certain embodiments, R¹ and R² are phenylalanine side chains and X¹ is methyl. In certain embodiments, R¹ and R² are phenylalanine side chains, R³ is hydrogen, and X¹ is methyl. In certain embodiments, R¹ is a phenylalanine side chain, R² is a tryptophan side chain, and X¹ is methyl. In certain embodiments, R¹ is a phenylalanine side chain, R² is a tryptophan side chain, R³ is hydrogen, and X¹ is methyl.

In certain embodiments, the compound of Formula (1) is a compound of Formula (1-aI):

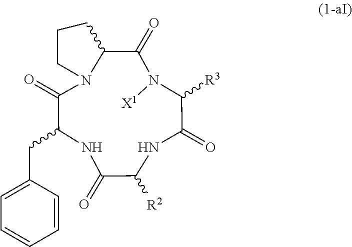

or a solvate or hydrate thereof;
wherein

each R² and R³ is independently hydrogen or an amino acid side chain; and

X¹ is substituted or unsubstituted alkyl.

In certain embodiments, R² is a phenylalanine side chain. In certain embodiments, R² is a tryptophan side chain. In certain embodiments, R² is an alanine side chain. In certain embodiments, R² is not hydrogen.

In certain embodiments, R³ is hydrogen. In certain embodiments, R³ is an alanine side chain.

In certain embodiments, X¹ is a substituted or unsubstituted C₁₋₆ alkyl. In certain embodiments, X¹ is a substituted or unsubstituted C₂₋₆ alkyl. In certain embodiments, X¹ is methyl. In certain embodiments, X¹ is ethyl.

In certain embodiments, R² is a phenylalanine side chain and X¹ is methyl. In certain embodiments, R² is a phenylalanine side chain, R³ is hydrogen, and X¹ is methyl. In certain embodiments R² is a tryptophan side chain and X¹ is methyl. In certain embodiments, R² is a tryptophan side chain, R³ is hydrogen, and X¹ is methyl.

In certain embodiments, the compound of Formula (1) is a compound of Formula (1-aII):

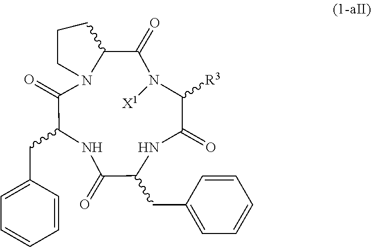

or a solvate or hydrate thereof;
wherein

R³ is hydrogen or an amino acid side chain; and

X¹ is substituted or unsubstituted alkyl.

In certain embodiments, R³ is hydrogen. In certain embodiments, R³ is an alanine side chain.

In certain embodiments, X¹ is a substituted or unsubstituted C₁₋₆ alkyl. In certain embodiments, X¹ is a substituted or unsubstituted C₂₋₆ alkyl. In certain embodiments, X¹ is methyl. In certain embodiments, X¹ is ethyl.

In certain embodiments, R³ is hydrogen and X¹ is methyl.

In certain embodiments, the compound of Formula (1) is a compound of Formula (1-aIII):

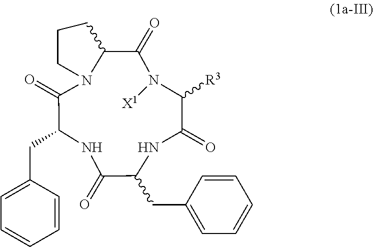

or a solvate or hydrate thereof;
wherein

R³ is hydrogen or an amino acid side chain; and

X¹ is substituted or unsubstituted alkyl.

In certain embodiments, R³ is hydrogen. In certain embodiments, R³ is an alanine side chain.

In certain embodiments, X¹ is a substituted or unsubstituted C₁₋₆ alkyl. In certain embodiments, X¹ is a substituted or unsubstituted C₂₋₆ alkyl. In certain embodiments, X¹ is methyl. In certain embodiments, X¹ is ethyl.

In certain embodiments, R³ is hydrogen and X¹ is methyl.

In certain embodiments, the compound of Formula (1) is a compound of Formula (1-aIV):

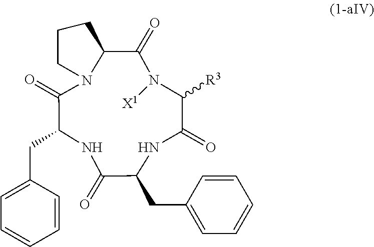

or a solvate or hydrate thereof;
wherein

R³ is hydrogen or an amino acid side chain; and

X¹ is substituted or unsubstituted alkyl.

In certain embodiments, R³ is hydrogen. In certain embodiments, R³ is an alanine side chain.

In certain embodiments, X¹ is a substituted or unsubstituted C₁₋₆ alkyl. In certain embodiments, X¹ is a substituted or unsubstituted C₂₋₆ alkyl. In certain embodiments, X¹ is methyl. In certain embodiments, X¹ is ethyl.

In certain embodiments, R³ is hydrogen and X¹ is methyl.

In certain embodiments, the compound of Formula I is:

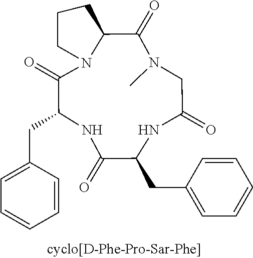 [SMILES: CN1CC(=O)N[C@@H](Cc2ccccc2)C(=O)N[C@H](Cc2ccccc2)C(=O)N2CCC[C@H]2C1=O]

or a solvate or hydrate thereof.

In some embodiments, one of the two phenyalanine side chain in a compound of Formula (1) is hydroxylated (i.e., the phenyalanine is a tyrosine analog). In some embodiments, one phenylalanine residue in a compound of Formula (1-aII) is hydroxylated. In some embodiments, hydroxylation of a compound of Formula (1-aII) produces an analog of Formula (1-aIIA):

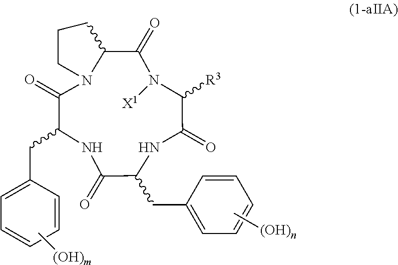

wherein m is 0 or 1; and n is 0 or 1. In some embodiments, when n and m are both not 1. In some embodiments, m is 1 and n is 0. In some embodiments, m is 0 is and n is 1.

In some embodiments, the hydroxyl (—OH) group is attached at the para position. In some embodiments, the hydroxyl (—OH) group is attached at the meta position.

In some embodiments, the compound is selected from:

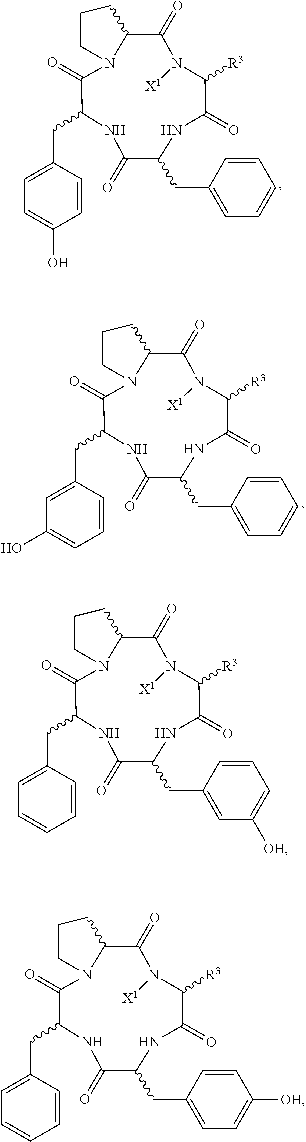

or a solvate or hydrate thereof.

Compounds delineated herein include salts, hydrates, solvates, and prodrugs thereof. In certain embodiments, compounds delineated herein include hydrate and solvates thereof. Compounds described herein may be derivatized to produce a salt form or prodrug form that may be more useful in one or more of the procedures and/or methods (e.g., methods of treatment) described herein. All compounds delineated in schemes herein are contemplated and included, whether intermediate or final compounds in a process.

Compounds of the invention can be made or modified by means known in the art of organic synthesis. Methods for optimizing reaction conditions, if necessary minimizing competing by-products, are known in the art. Additional reaction schemes, optimization, scale-up, and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. For example, compounds of formulae herein can be made using methodology known in the art, including Eans, S. O., Ganno, M. L., Reilley, K. J., Patkar, K. A., Senadheera, S. N., Aldrich, J. V., and McLaughlin, J. P. (2013) The macrocyclic tetrapeptide [D-Trp]CJ-15,208 produces short-acting κ opioid receptor antagonism in the CNS after oral administration. Br J Pharmacol 169, 426-436; Aldrich, J. V., Senadheera, S. N., Ross, N. C., Reilley, K. A., Ganno, M. L., Eans, S. E., Murray, T. F., and McLaughlin, J. P. (2014) Alanine analogues of [D-Trp]CJ-15,208: Novel opioid activity profiles and prevention of drug- and stress-induced reinstatement of cocaine-seeking behavior, British Journal of Pharmacology 171, 3212-3222; and Ross, N. C., Kulkarni, S. S., McLaughlin, J. P., and Aldrich, J. V. (2010) Synthesis of CJ-15,208, a novel κ-opioid receptor antagonist Tetrahedron Lett 51, 5020-5023; and Aldrich, J. V. Kulkarni, S. S., Senadheera, S. N.; Ross, N.C., Reilley, K. J., Eans, S., Ganno, M. L., Murray, T. F., and McLaughlin, J. P. et al. (2011) Unexpected opioid activity profiles of analogs of the novel peptide kappa opioid receptor ligand CJ-15,208. Chem Med Chem 6, 1739-1745; Aldrich et al. Journal of Natural Products, 76, 433-438 (2013); each of which is incorporated herein by reference in its entirety.

The compounds of the formulae herein can be synthesized using methodology similar to that shown in the following schemes.

Scheme I illustrates the synthesis of cyclo[D-Phe-Pro-Sar-Phe] (G) from the linear peptide F by Fmoc-based solid phase peptide synthesis (SPPS) on 2-chlorotrityl resin. The 2-chlorotrityl resin was loaded with 4 equivalents of Fmoc-protected phenylalanine (Fmoc-Phe-OH) using 8 equivalents N,N-diisopropylethylamine (DIEA) over 6 hours to give A. Fmoc quantitation was performed to determine loading efficiency. The remainder of the linear peptide was synthesized according to standard coupling and deprotection protocols. Deprotection of the terminal amino acid is carried out in the presence of a mild base, e.g., 20% 4-methylpiperidine, and Fmoc-protected sarcosine (Fmoc-Sar-OH) coupled to the resulting amine using 1-hydroxybenzotriazole (HOBt), benzotriazol-1-yl-oxy-tris(pyrrolidino)phosphonium hexafluorophosphate (PyBOP), and DIEA to yield B. Deprotection of the terminal amino acid is carried out in the presence of a mild base, e.g., 20% 4-methylpiperidine, and Fmoc-protected proline (Fmoc-Pro-OH) coupled to the resulting amine using 1-hydroxybenzotriazole (HOBt), benzotriazol-1-yl-oxy-tris(pyrrolidino)phosphonium hexafluorophosphate (PyBOP), and DIEA to yield C. Deprotection of the terminal amino acid is carried out in the presence of a mild base, e.g., 20% 4-methylpiperidine, and Fmoc-protected D-phenylalanine (Fmoc-Phe-OH) coupled to the resulting amine using 1-hydroxybenzotriazole (HOBt), benzotriazol-1-yl-oxy-tris(pyrrolidino)phosphonium hexafluorophosphate (PyBOP), and DIEA to yield D. A final deprotection step is carried out in the presence of a mild base, e.g., 20% 4-methylpiperidine to yield E with a free amino-terminus. At any step in the reaction, the D- or L-isomer of the amino acid can be added, depending on the desired stereochemistry of the final product. Cleavage of the linear peptide from the 2-chlorotrityl resin is achieved under acidic conditions in the presence of 1% trifluoroacetic acid (TFA) and solvent is removed to yield the free linear peptide F. The linear peptide F was lyophilized prior to cyclization. Cyclization of the linear peptide to yield the macrocyclic tetrapeptide G was carried out as follows. The linear peptide precursor (25 mM, 0.5 equiv) in DMF was added dropwise at a rate of 1 mL/h to a solution of 1.5 equivalents of 0.9 mM O-(7-azabenzotriazol-1-yl)-N,N,N′,N′-tetramethyluronium hexafluorophosphate (HATU) and 8 equivalents of DIEA (8 equiv) in DMF. Following addition of the peptide, a second portion of HATU (1.5 equiv) was added directly to the solution, and additional linear peptide (25 mM, 0.5 equiv) in DMF was added dropwise at a rate of 1 mL/h. The reaction was then allowed to stir for 12-30 h. The temperature was increased to 40° C. and allowed to stir for an additional 24 h. The solvent was evaporated, the residue dissolved in ethyl acetate and washed three times each with 1 N citric acid, saturated sodium bicarbonate, and brine. The organic solution was dried over magnesium sulfate and solvent evaporated. The resulting product was purified by normal phase flash chromatography (50-100% ethylacetate in hexane, then 0-5% MeOH) and lyophilized. The macrocyclic tetrapeptide G was characterized by mass spectrometry and HPLC (Method A: 15-55% acetonitrile/water +0.1% TFA over 40 min, 214 nm; Method B: 30-70% methanol/water +0.1% TFA over 40 min, 230 nm). HPLC retention time was 11.2 min (Method A) and 9.7 min (Method B); M+1=463.2 m/z (expected=463.2 m/z), M+Na=485.2 m/z (expected=485.2 m/z). No side chain protection was necessary for cyclization for this peptide; however Fmoc-protected amino acids with side chain protecting groups could also be substituted for any of the amino acids in the synthesis of G. Cyclization conditions have been optimized to minimize the formation of the cyclic dimer and maximize the yield of the macrocyclic tetrapeptide and previously published [Kulkarni, S. S., Ross, N. C., McLaughlin, J. P., and Aldrich, J. V. (2009) Synthesis of cyclic tetrapeptide CJ 15,208: a novel kappa opioid receptor antagonist Adv Exp Med Biol 611, 269-270; Ross, N. C., Kulkarni, S. S., McLaughlin, J. P., and Aldrich, J. V. (2010) Synthesis of CJ-15,208, a novel κ-opioid receptor antagonist Tetrahedron Lett 51, 5020-5023; Aldrich, J. V., Senadheera, S. N., Ross, N. C., Ganno, M. L., Eans, S. O., and McLaughlin, J. P. (2013) The macrocyclic peptide CJ-15,208 is orally active and prevents reinstatement of extinguished cocaine-seeking behavior J Nat Prod 76, 433-438; Senadheera, S. N., Kulkarni, S. S., McLaughlin, J. P., and Aldrich, J. V. (2011) Improved synthesis of CJ-15,208 isomers and their pharmacological activity at opioid receptors In Peptides: Building Bridges. Proceedings of the 22nd American Peptide Symposium, Lebl, M., Ed. American Peptide Society: San Diego, Calif., pp 346-347]. The macrocyclic tetrapeptides can be further purified by normal phase flash column chromatography as previously described by published procedures [Senadheera, S. N., Kulkarni, S. S., McLaughlin, J. P., and Aldrich, J. V. (2011) Improved synthesis of CJ-15,208 isomers and their pharmacological activity at opioid receptors In Peptides: Building Bridges. Proceedings of the 22nd American Peptide Symposium, Lebl, M., Ed. American Peptide Society: San Diego, Calif., pp 346-347]. Routine preparation of 500-600 mg of analogs can be achieved with this methodology, and the synthesis and purification can be scaled up if necessary.

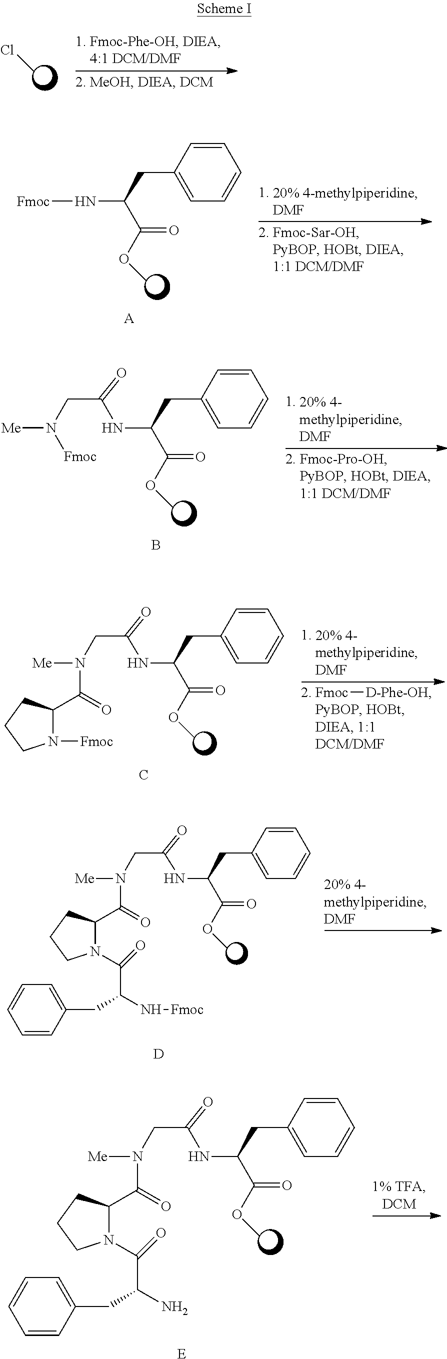

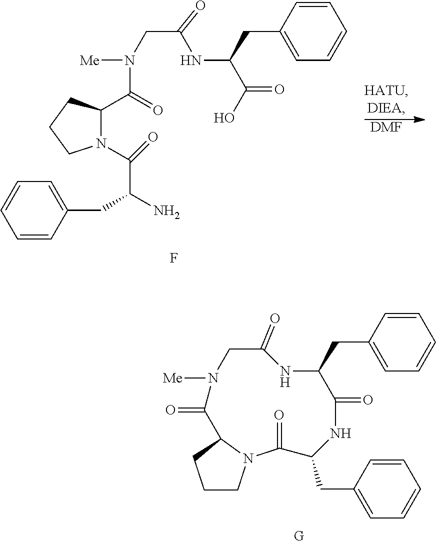

### Pharmaceutical Compositions

Also provided herein are pharmaceutical compositions comprising a compound of Formula (1) and optionally a pharmaceutically acceptable excipient, adjuvant, or carrier. In certain embodiments, the compound is present in an effective amount (e.g., a therapeutically effective amount or a prophylactically effective amount) in the pharmaceutical composition.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In certain embodiments, the compounds are formulated for oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for intravenous administration.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject, any two doses of the multiple doses include different or substantially the same amounts of a compound described herein. In certain embodiments, when multiple doses are administered to a subject, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, when multiple doses are administered to a subject, the frequency of administering the multiple doses to the subject or applying the multiple doses to the tissue or cell is four doses a day, three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, the frequency of administering the multiple doses to the subject is four doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in activating or reducing (e.g., antagonizing) the activity of an opioid receptor in a subject or cell), improve bioavailability, improve safety, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents (e.g., therapeutic agent) include, but are not limited to, anti-proliferative agents, anti-cancer agents, anti-inflammatory agents, anti-depressant agents, immunosuppressants, and pain-relieving agents. In certain embodiments, the anti-depressant agent is selected from selective serotonin reuptake inhibitors (SSRIs), serotonin-norepinephrine reuptake inhibitors (SNRIs), tricyclic antidepressants (TCAs), monoamine oxidase inhibitors (MAOIs), reversible inhibitors of monoamine oxidase A (RIMAs), tetracyclic antidepressants (TeCAs), and noradrenergic and specific serotonergic antidepressant (NaSSAs). In certain embodiments, the anti-inflammatory agent is a nonsteroidal anti-inflammatory drug (NSAID).

### Methods of Use

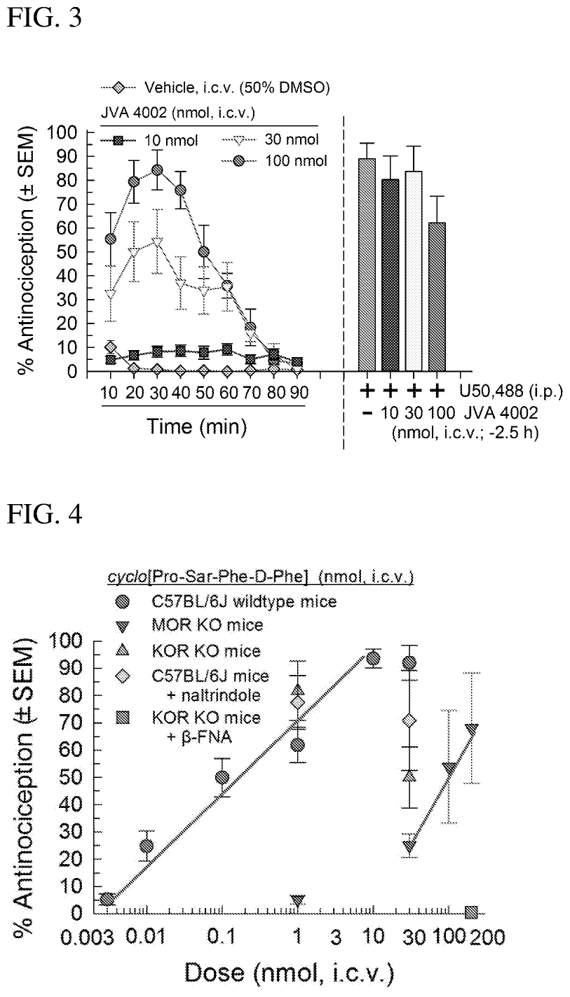

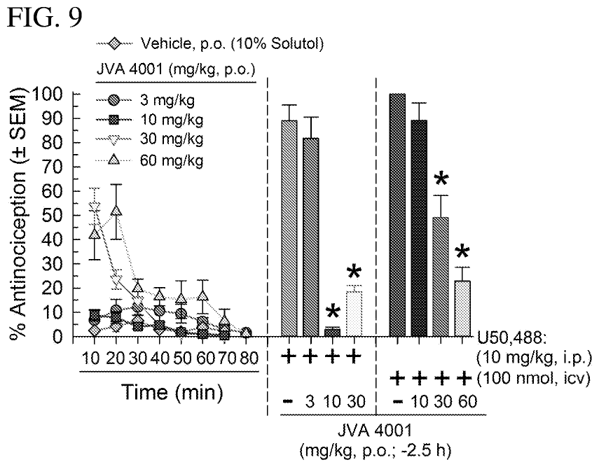

In some aspects, the compounds of Formula (1) described herein modulate one or more opioid receptors. For example, without wishing to be bound by any particular theory, the macrocylic tetrapeptide cyclo[D-Phe-Pro-Sar-Phe) shows selective antagonistic activity against the kappa opioid receptor (KOR). In addition, antinociception is mediated by the mu opioid receptor, indicating that this tetrapeptide also functions as a mu opioid receptor agonist. The macrocyclic tetrapeptide cyclo[D-Phe-Pro-Sar-Phe] is a selective and extremely potent (1 pmol i.c.v.) antagonist at KOR that at 100-fold higher doses also exhibits antinociception that is mediated by mu opioid receptors at higher doses (EC₅₀=0.11 nmol i.c.v.). This compound prevents both stress- and cocaine-induced reinstatement of cocaine-seeking behavior in a time-dependent manner. Following oral administration it dose-dependently antagonizes antinociception mediated by a centrally administered KOR agonist, suggesting it crosses the blood-brain barrier, and also exhibits transient antinociception. Noteably, at higher doses, JVA-4001 shows characteristics of both a mu opioid receptor agonist and a kappa opioid receptor agonist (FIGS. 8A-B). In addition, these novel and beneficial properties are unique to compounds of Formula (1), as analogs examined, such as cyclo[D-Phe-Pro-D-NMe-Ala-Phe] (also referred to as JVA-4002 in FIG. 3), did not exhibit KOR antagonist activity. However, cyclo[D-Phe-Pro-D-NMe-Ala-Phe] does show antinoiception at higher dosages (FIG. 3). This D-NMe-Ala analog is also metabolized by mouse liver microsomes more rapidly (t_{1/2}=12 minutes) than cyclo[D-Phe-Pro-Sar-Phe] (t_{1/2}=60 minutes).

Thus, in one aspect, the invention provides a compound that is an opioid receptor agonist. In some embodiments, the compound is an agonist of a mu opioid receptor. In some embodiments, the compound is an agonist of a mu opioid receptor and an agonist of a kappa opioid receptor.

In another aspect, the invention provides a compound that is an opioid receptor antagonist. In some embodiments, the compound is an antagonist of a kappa opioid receptor.

In a further embodiment, the invention provides a compound that produces its activity through more than one opioid receptor. The compounds may modulate the opioid receptors by a similar mechanism, e.g., the compound is an agonist of a first and a second opioid receptor, or by different mechanisms, e.g., the compound is an antagonist of a first opioid receptor and an agonist of a second opioid receptor. In certain embodiments, the compound is an antagonist of a first opioid receptor and an agonist of a second opioid receptor. In some embodiments, the compounds may modulate the same receptor by different mechanisms at different times after dosage, e.g., the compound may be a first opioid receptor agonist initially upon administration and a first opioid receptor antagonist at later times post-administration (i.e., the compound displays mixed agonist/antagonist activity). In some embodiments, the first opioid receptor is a kappa opioid receptor, mu opioid receptor, or delta opioid receptor. In some embodiments, the second opioid receptor is a kappa opioid receptor, mu opioid receptor, or delta opioid receptor. In some embodiments, the first opioid receptor is a kappa opioid receptor. In some embodiments, the second opioid receptor is a mu opioid receptor.

Thus, in one aspect, provided herein is a method for modulating an opioid receptor.

In a further aspect, provided herein is a method for reducing or preventing the activation (e.g., biological output) of an opioid receptor, wherein the method comprises contacting the opioid receptor with an effective amount of a compound of Formula (1). In some embodiments, the opioid receptor is a kappa opioid receptor, a mu opioid receptor, or a delta opioid receptor. In some embodiments, the opioid receptor is a kappa opioid receptor.

In a further aspect, provided herein is a method for activating or increasing the activity (e.g., biological output) of an opioid receptor, wherein the method comprises contacting the opioid receptor with an effective amount of a compound of Formula (1). In some embodiments, the opioid receptor is a kappa opioid receptor, a mu opioid receptor, or a delta opioid receptor. In some embodiments, the opioid receptor is a mu opioid receptor.

In some embodiments, the opioid receptor is in vitro. In some embodiments, the opioid receptor is in vivo.

In another aspect, provided herein is a method for reducing or preventing nociception (e.g., promoting antinociception), where in the method comprises administering to the subject an effective amount of a compound of Formula (1). In certain embodiments, the subject is a non-human mammal. In certain embodiments, the subject is a human. In certain embodiments, an effective amount is a therapeutically effective amount or a prophylactically effective amount.

In another aspect, provided herein is a method of treating a subject with a disease, disorder, or symptoms thereof, wherein the method comprises administering to the subject an effective amount of a compound of Formula (1). In certain embodiments, the subject is a non-human mammal. In certain embodiments, the subject is a human. In certain embodiments, an effective amount is a therapeutically effective amount or a prophylactically effective amount.

In certain embodiments, the disorder is a neurological disorder. In certain embodiments, the neurological disorder is addiction. In certain embodiments, the addiction is an alcohol addiction. In certain embodiments, the addiction is a drug addiction. In certain embodiments, the addiction is an opioid addiction. In certain embodiments, the addiction is a cocaine addiction. In certain embodiments, the subject has previously suffered from an addiction and is more likely to relapse.

In certain embodiments, the disorder is an opioid receptor mediated disorder. In certain embodiments, the opioid receptor mediated disorder is stress-induced reinstatement of drug-seeking behavior. In certain embodiments, the stress-induced reinstatement of drug-seeking behavior is stress-induced reinstatement of cocaine-seeking behavior. In certain embodiments, the opioid receptor mediated disorder is drug-induced reinstatement of drug-seeking behavior. In certain embodiments, the drug-induced reinstatement of drug-seeking behavior is cocaine-induced reinstatement of cocaine-seeking behavior.

In certain embodiments, the disorder is an opioid receptor mediated disorder. In certain embodiments, the opioid receptor mediated disorder is stress-induced reinstatement of drug-seeking behavior. In certain embodiments, the stress-induced reinstatement of drug-seeking behavior is stress-induced reinstatement of cocaine-seeking behavior. In certain embodiments, the stress-induced reinstatement of drug-seeking behavior is stress-induced reinstatement of ethanol-seeking behavior. In certain embodiments, the opioid receptor mediated disorder is drug-induced reinstatement of drug-seeking behavior. In certain embodiments, the drug-induced reinstatement of drug-seeking behavior is cocaine-induced reinstatement of cocaine-seeking behavior. In certain embodiments, the drug-induced reinstatement of drug-seeking behavior is opioid-induced reinstatement of opioid-seeking behavior.

In certain embodiments, the disorder is a psychiatric disorder. A psychiatric disorder includes, for example, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder. In certain embodiments, the substance-related disorder is a substance abuse. In certain embodiments, the substance abuse is a cocaine abuse. In certain embodiments, the substance abuse is alcohol abuse.

In certain embodiments, the disorder is a psychiatric disorder. A psychiatric disorder includes, for example, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder. In certain embodiments, the substance-related disorder is a substance abuse. In certain embodiments, the substance abuse is a cocaine abuse. In certain embodiments, the substance abuse is alcohol abuse. In certain embodiments, the substance abuse is opioid abuse.

In certain embodiments, the disorder is a painful condition or symptoms associated with a painful condition. In certain embodiments, the painful condition is induced (i.e., caused) by nociceptive pain. In certain embodiments, the nociceptive pain is the caused by a chemical, mechanical, or thermal stimulus. In certain embodiments, the painful condition is pain associated with withdrawal from an addiction. In certain embodiments, the addiction is an alcohol addiction. In certain embodiments, the addiction is a drug addiction. In certain embodiments, the drug addiction is an opioid addiction. In certain embodiments, the drug addiction is a cocaine addiction.

A further aspect presents a method of administering a therapeutically effective amount of a compound of the invention to a subject in need of an analgesic. The phrase “in need of analgesic” encompasses any disease or disorder that results in pain in the subject.

In certain embodiments, the methods of the invention include administering to a subject an effective amount of a compound described herein in combination with another pharmaceutically active compound. Pharmaceutically active compounds that may be used can be found in Harrison's Principles of Internal Medicine, Nineteenth Edition, Eds. T. R. Harrison et al. McGraw-Hill N.Y., N.Y.; and the Physicians Desk Reference 71st Edition 2017, Oradell N.J., Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired.

Compounds determined to be effective for the prevention or treatment of neurological disorders, opioid receptor mediated disorders, psychiatric disorders, or painful conditions in animals, e.g., primates, and rodents (e.g., mice), may also be useful in treatment of these disorders or conditions in humans. Those skilled in the art of treating neurological disorders, opioid receptor mediated disorders, psychiatric disorders, or painful conditions in humans will know, based upon the data obtained in animal studies, the dosage and route of administration of the compound to humans. In general, the dosage and route of administration in humans is expected to be similar to that in animals.

The identification of those patients who are in need of treatment for neurological disorders, opioid receptor mediated disorders, psychiatric disorders, or painful conditions is well within the ability and knowledge of one skilled in the art. Certain methods for identification of patients which are at risk of developing the disorders or conditions described herein which can be treated by the subject methods are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a neurological disorder, opioid receptor mediated disorder, psychiatric disorder, or painful condition by methods well known in the art (e.g., antinociceptive testing) and then administering a therapeutically effective amount of a compound of any formula herein or otherwise described herein according to the invention to the subject. After an appropriate period of time after the administration of the compound (e.g., 1 hour, 2 hours, 3 hours, 4 hours, 5 hours, 6 hours, 7 hours, 8 hours, 9 hours, 10 hours, 11 hours, 12 hours, 1 day, etc.), the extent of the opioid receptor mediated disorder is determined again. The modulation (e.g., decrease or increase) of the activity of the opioid receptor of the opioid receptor mediated disorder indicates efficacy of the treatment. The extent of modulation of the activity of the opioid receptor may be determined periodically throughout treatment. For example, the extent of modulation of the activity of the opioid receptor may be checked every few hours, days or weeks to assess the further efficacy of the treatment. When the compound is an antagonist, a decrease of the activity of the opioid receptor in the opioid receptor mediated disorder indicates that the treatment is efficacious. When the compound is an agonist, an increase of the activity of the opioid receptor in the opioid receptor mediated disorder indicates that the treatment is efficacious. The method described may be used to screen or select patients that may benefit from treatment with an agonist and/or antagonist of an opioid receptor.

### EXAMPLES

The invention is further illustrated by the following examples which are intended to illustrate but not limit the scope of the invention.

The compounds of the invention can be evaluated for their opioid activity in vitro and in vivo through a variety of assays known in the field. The following examples provide exemplary protocols for evaluating the opioid activity of the compounds of the invention.

### Example 1: In Vitro Metabolism and Stability Studies

The in vitro stability of the compounds of the invention in mouse hepatic microsomes can be examined. Following incubation with the compound for various times at 37° C., the proteins are precipitated with MeCN, and the samples are centrifuged and analyzed by LC-MS/MS. The apparent t_{1/2} can be calculated for disappearance of the compound from the microsomes. In cases where appreciable metabolism appears to be occurring, metabolites can be characterized by LC-MS/MS. The stability of selected compounds of the invention can also be analyzed in mouse brain homogenate using similar procedures.

The metabolic stability of the macrocyclic tetrapeptide cyclo[D-Phe-Pro-Sar-Phe] was evaluated in vitro in mouse liver microsomes and has an apparent t_{1/2} of ˜60 minutes. This is an improvement over the in vitro stability of [D-Trp]CJ-15,208 (t_{1/2}=11 minutes).

### Example 2: In Vivo Pharmacokinetic Analysis Using LC-MS/MS

The compounds of the invention can be administered to rats and mice in different dosages. Blood samples (0.25 mL) can be obtained from rats or mice at various time points, and the amount of compound in the plasma can be determined by LC-MS/MS [Khaliq, T., Williams, T. D., Senadheera, S. N. and Aldrich, J. V. (2016) Development of a robust, sensitive and selective liquid chromatography-tandem mass spectrometry assay for the quantification of the novel macrocyclic peptide kappa opioid receptor antagonist [D-Trp]CJ-15,208 in plasma and application to an initial pharmacokinetic study J. Chromatogr. B, 1028, 11-15]. These studies provide basic PK parameters (AUC, Cₘₐₓ, t_{1/2}), and the PK data can be analyzed using WinNonlin software. The blood samples can also be monitored for the presence of any metabolites identified in the in vitro analysis in hepatic microsomes described above. The PK parameters of selected compounds of the invention can also be obtained using LC-MS/MS following oral administration to assess oral bioavailability.

### Example 3: Intracerebroventricular (i.c.v.) and Intraperitoneally (i.p.) Administration Techniques

The intracerebroventricular (i.c.v.) injections of the compounds of the invention are made directly into the lateral ventricle (e.g. of a mouse) according to the modified method as published [Haley, T. J. and McCormick, W. G. (1957) Pharmacological effects produced by intracerebral injections of drugs in the conscious mouse Br J Pharmacol Chemother 12, 12-15]. Briefly, the volume of injections is 5 μL. The mouse is lightly anaesthetized with isoflurane, an incision made in the scalp, and the injection made 2 mm lateral and 2 mm caudal to bregma at a depth of 3 mm using a 10 μL Hamilton syringe.

Sterile isotonic saline (0.9%), 50% dimethyl sulfoxide (DMSO) or 10% Solutol were used to dissolve compounds to desired concentrations for testing. cyclo[D-Phe-Pro-Sar-Phe] was primarily administered through the intracerebroventricular (i.c.v.) route in a volume of 5 μl directly into the lateral ventricle as described previously, or intraperitoneally (i.p.) in a volume of 250 μL/25 g body weight [Ross, N.C., Reilley, K. J., Murray, T. F., Aldrich, J. V. and McLaughlin, J. P., 2012. Novel opioid cyclic tetrapeptides: Trp isomers of CJ-15,208 exhibit distinct opioid receptor agonism and short-acting κ opioid receptor antagonism. Br. J. Pharmacol., 165(4b), 1097-1108]. Additional drugs were administered i.p. in a volume of 250 μl/25 g body weight.

### Example 4: 55° C. Warm-Water Tail-Withdrawal Assay

Antioceptive testing in the presence of compounds of the invention can be conducted in vivo using a 55° C. warm-water tail-withdrawal assay as published [McLaughlin, J. P., Hill, K. P. Jiang, Q., Sebastian, A., Archer, S., and Bidlack, J. M. (1999) Nitrocynnamoyl and chlorocinnamoyl derivatives of dihydrocodeinone: in vitro and in vivo characterization of mu-selective agonist and antagonist activity J Pharmacol Exp Ther 289, 304-311]. Generally, the mice used for this assay are C57Bl/6 mice. Briefly, warm (55° C.) water in a 2 L heated water bath is used as the thermal nociceptive stimulus, with the latency of the mouse to withdraw its tail from the water taken as the endpoint. After determination of baseline tail-withdrawal latencies, mice are administered a graded dose of a compound of the invention i.c.v. where the compounds of the invention are administered in 50% DMSO in sterile saline (0.9%). Alternatively, a compound of the invention is administered to mice systemically (e.g., intraperitoneally, subcutaneously, or orally) in saline solution which typically contains 10% Solutol HS 15. To determine agonist activity, the tail-withdrawal latency is determined repeatedly every 10 min following administration of a compound of the invention for 1 h or until latency returns to baseline values. A cut-off time of 15 seconds can be used in this study; if the mouse fails to display a tail-withdrawal response during that time, the tail is removed from the water and the animal is assigned a maximal antinociceptive score of 100%. At each time point, antinociception can be calculated according to the following formula:

% antinociception=100×(test latency−control latency)/(15−control latency)

Student's t-tests and ANOVA with Tukey's HSD post hoc tests can be used to compare baseline and post-treatment tail-withdrawal latencies and to determine statistical significance for all tail-withdrawal data. Generally, independent experiments from several (e.g., seven to ten) mice are conducted and analyzed to increase the statistical significance of the tail-withdrawal data. Potency can be quantified by calculating ED₅₀ values with standard software known in the art (e.g., Prism 6.0 software, GraphPad Software, La Jolla, Calif., USA).

To determine antagonist activity, mice are pretreated with the compound of the invention 80 min-2.5 h before administration of the μ opioid receptor-preferring agonist morphine (10 mg·kg⁻¹, i.p.), κ opioid receptor-selective agonist U50,488 (10 mg·kg⁻¹, i.p.) or δ opioid receptor-selective agonist SNC-80 (100 nmol, i.c.v.); the agonists are administered using sterile saline (0.9%) as the vehicle, except for SNC-80 which is dissolved in 35% DMSO in sterile saline (0.9%). Antinociception produced by these established agonists is then measured 40 min after their administration. To determine the duration of κ opioid receptor antagonist activity, additional mice can be pretreated for 3.7-47.3 h before the administration of U50,488 as described previously.

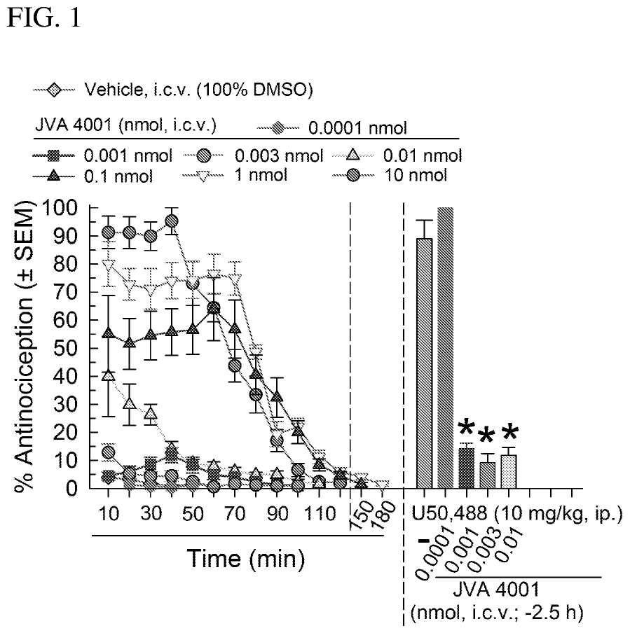

The cyclic peptide cyclo[D-Phe-Pro-Sar-Phe] (code number JVA-4001) was evaluated in vivo for antinociceptive activity and KOR antagonism in this assay. This peptide exhibited significant time- and dose-dependent antinociceptive activity following i.c.v. administration (p<0.0001), with an ED₅₀ (and 95% confidence interval) of 0.11 (0.05-0.21) nmol (FIG. 1, left panel), 21.4-fold more potent than morphine [2.35 (1.13-5.03) nmol]. Antinociception lasted up to 80 min after administration of a 10 nmol dose (p=0.01).

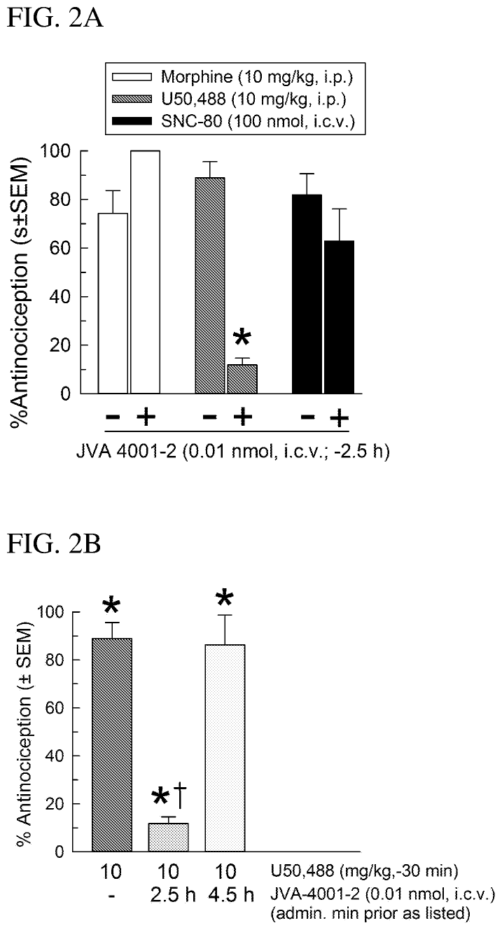

cyclo[D-Phe-Pro-Sar-Phe] demonstrated even more potent KOR antagonism, with a significant blockade of U50,488-induced antinociception with a 2.5 h pretreatment of 1 pmol (p<0.0001) (FIG. 1, right panel). cyclo[D-Phe-Pro-Sar-Phe] significantly antagonized KOR in a dose-dependent manner after i.c.v. administration; FIG. 1. The KOR antagonism produced by a single dose (10 pmol) of cyclo[D-Phe-Pro-Sar-Phe] was short-acting, lasting at least 2.5 h, but less than 4.5 h (p<0.0001; FIG. 2B).

cyclo[D-Phe-Pro-Sar-Phe] antagonism was KOR selective (FIG. 2A). Pretreatment with cyclo[D-Phe-Pro-Sar-Phe] (0.01 nmol, i.c.v.) 150 min prior to agonist administration demonstrated significant antagonism of only the KOR-selective agonist U50,488 (p<0.0001; FIG. 2A). A 2.5 h pretreatment with a much higher dose (30 nmol i.c.v.) of cyclo[D-Phe-Pro-Sar-Phe] did not significantly antagonize morphine- or SNC80-mediated antinociception (100±0% and 87.1±7.7%, respectively), confirming KOR-selective antagonist activity.

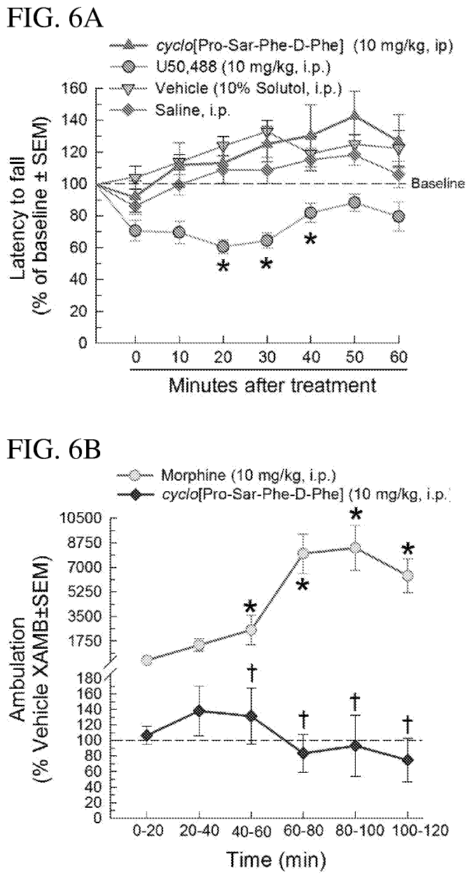

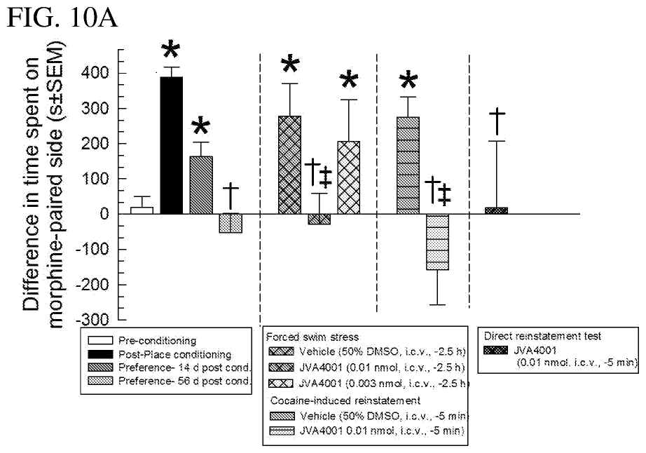

We next determined that cyclo[D-Phe-Pro-Sar-Phe] demonstrated dose-dependent transient antinociceptive activity and KOR antagonism after peripheral administration through both the intraperitoneal (i.p., FIGS. 5A-B) and oral (p.o., FIG. 9) routes. Comparison of the ED₅₀ values produced by cyclo[D-Phe-Pro-Sar-Phe] demonstrated potency equivalent to or slightly greater than morphine when administered through the i.p. route (EC₅₀=1.91 (0.40-3.54) vs. 3.91 (2.92-5.17) mg/kg, respectively). Of interest, cyclo[D-Phe-Pro-Sar-Phe] demonstrated KOR antagonism 2.5 h after oral administration (FIG. 9), suggesting this analog was absorbed from the gastrointestinal tract. Moreover, orally administered cyclo[D-Phe-Pro-Sar-Phe] dose-dependently antagonized i.c.v. U50,488, further suggesting that cyclo[D-Phe-Pro-Sar-Phe] crosses the blood-brain barrier to penetrate the central nervous system. These results are consistent with the action of the original macrocyclic tetrapeptide CJ-15,208 (see, Aldrich, J. V., Senadheera, S. N., Ross, N. C., Ganno, M. L., Eans, S. O., and McLaughlin, J. P. (2013) The macrocyclic peptide natural product CJ-15,208 is orally active and prevents reinstatement of extinguished cocaine-seeking behavior, J Nat Prod 76, 433-438).

### Example 5: Opioid Receptor Specificity

To determine the opioid receptor selectivity of the agonist activity of the compound of the invention, MOR knock-out (KO) mice and KOR KO mice are treated with the compound, with antinociceptive testing 40 min later. The 55° C. warm-water tail-withdrawal test can be used to determine opioid receptor specificity in mice carrying these gene knockouts. Additional wild-type mice can be pretreated 30 min prior to the administration of the compound of the invention with the DOR-selective antagonist naltrindole (20 mg·kg⁻¹, i.p. or 0.5 mg/kg, s.c.), with antinociceptive testing 40 min later; the antagonist is administered using sterile saline (0.9%) as the vehicle.

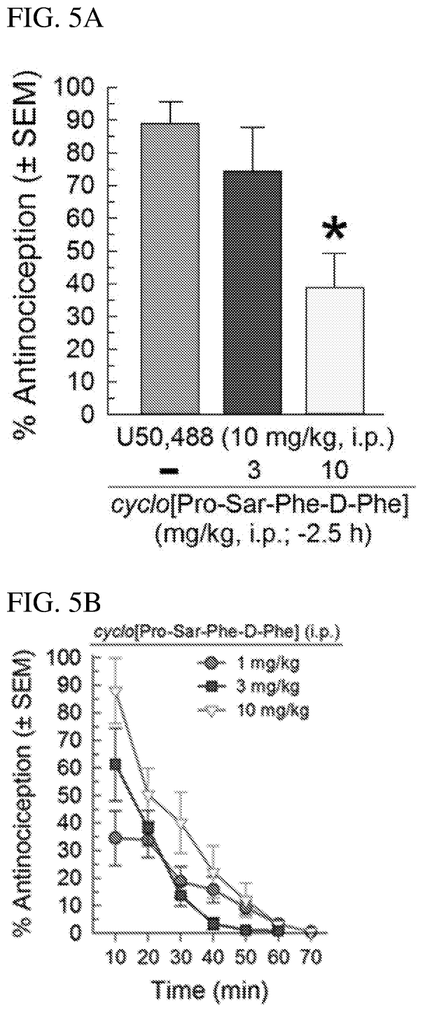

The opioid receptor selectivity of cyclo[D-Phe-Pro-Sar-Phe]-induced antinociception was examined in opioid receptor gene-disrupted (KO) mice or wild-type mice treated with the DOR-selective antagonist naltrindole (FIG. 4). Antinociception produced by a 1 nmol dose of cyclo[D-Phe-Pro-Sar-Phe] was significantly impaired only in MOR KO mice (p<0.0001). At a 30-fold higher dose (30 nmol, i.c.v.) of cyclo[D-Phe-Pro-Sar-Phe], the contribution of MOR to antinociception remained significant (p=0.0²) but somewhat reduced, with evidence of additional mediation by KOR; a dose-response curve in MOR KO mice yielding an ED₅₀ (and 95% C.I.) value of 58.2 (17.4-558) nmol, i.c.v. The antinociception produced by a high test dose (200 nmol, i.c.v.) of cyclo[D-Phe-Pro-Sar-Phe] was completely inhibited by pretreatment of KOR KO mice with the MOR-selective antagonist β-FNA, suggesting that cyclo[D-Phe-Pro-Sar-Phe] acts primarily as a potent MOR agonist with additional, modest KOR agonism.

### Example 6: Conditioned Place Preference (CPP)

An automated, balanced three-compartment place conditioning apparatus (San Diego Instruments, San Diego, Calif., USA) and a 2- or 4-day counterbalanced morphine-place conditioning design was used, similar to methods previously described [Eans, S. O., Ganno, M. L., Reilley, K. J., Patkar, K. A., Senadheera, S. N., Aldrich, J. V. and McLaughlin, J. P., 2013. The macrocyclic tetrapeptide [D-Trp] CJ-15,208 produces short-acting κ opioid receptor antagonism in the CNS after oral administration. British Journal of Pharmacology, 169(2), 426-436]. The amount of time subjects spent in each of the three compartments was measured over a 30 min testing period. Prior to place conditioning, the animals did not demonstrate significant differences in their time spent exploring the outer left (639±10.0 s) versus right (610±9.63 s) compartments (p=0.10). To perform place conditioning, mice were administered vehicle (0.9% saline) and consistently confined in a randomly assigned outer compartment: half of each group in the right chamber, half in the left chamber. Four hours later, mice were administered test compound and confined to the opposite compartment for 40 min.

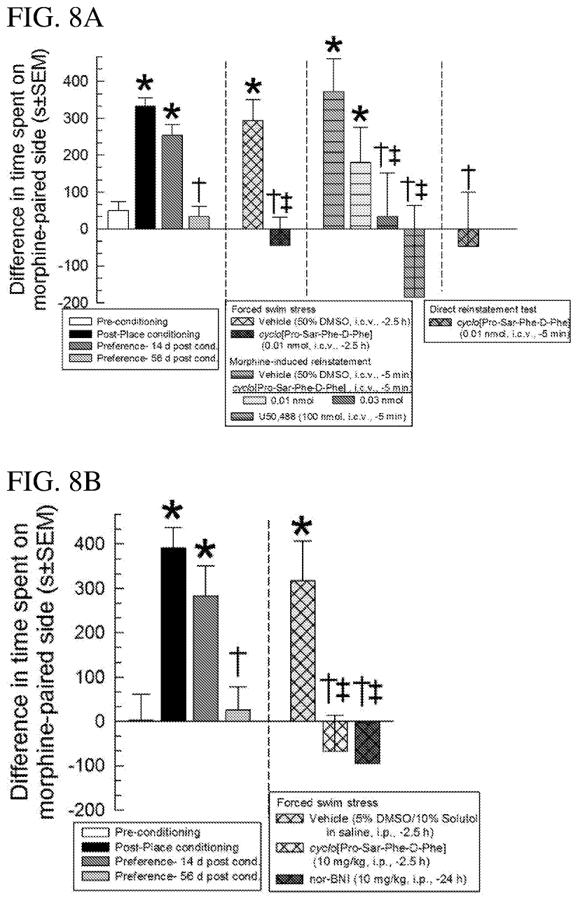

To determine if cyclo[D-Phe-Pro-Sar-Phe] (0.1, 1, or 10 nmol, i.c.v.) produced CPP or conditioned place aversion (CPA) mice were place conditioned in this way for two days, with final testing on the fourth day, as this has been shown to produce dependable morphine CPP and U50,488-induced CPA [Haji, A. and Takeda, R., 2001. Effects of a κ-receptor agonist U-50488 on bulbar respiratory neurons and its antagonistic action against the μ receptor-induced respiratory depression in decerebrate cats. The Japanese Journal of Pharmacology, 87(4), 333-337]. To assess for potential rewarding or aversive behavior without potential confounds related to compound distribution to the brain after peripheral administration, cyclo[D-Phe-Pro-Sar-Phe] was evaluated in the place conditioning paradigm after central administration. Data are plotted as the difference in time spent in the compound- and vehicle-paired sides. By convention, a positive value represents a conditioned preference for the compound-paired side. CPA, where the animals avoid the compound-paired compartment and spend a significantly greater time in the saline-paired compartment than initially demonstrated, may be detected in this study. Following a two-day place-conditioning paradigm, mice conditioned with morphine demonstrated a significant place preference response (p<0.001; FIG. 7), whereas U50,488 produced significant CPA (p=0.009). In contrast, mice conditioned with cyclo[D-Phe-Pro-Sar-Phe] (0.1, 1, or 10 nmol, i.c.v.) demonstrated neither a significant preference for, nor aversion to, the drug paired chamber.

### Example 7: Cocaine, Morphine, and Ethanol Conditioned Place Preference (CPP), Extinction, and Reinstatement

### Cocaine:

Mice can be conditioned based on a previously established cocaine CPP paradigm as published [Carey, A. N., Borozny, K., Aldrich, J. V., and McLaughlin, J. P., (2007) Reinstatement of cocaine place-conditioning prevented by the peptide kappa-opioid receptor antagonist, Arodyn Eur J Pharmacol 569, 84-89; Aldrich, J. V., Patkar, K. A., McLaughlin, J. P. (2009) Zyklophin, a systemically active selective kappa opioid receptor peptide antagonist with short duration of action Proc Natl Acad Sci USA 106, 18396-18401; Eans, S. O., Ganno, M. L., Reilley, K. J., Patkar, K. A., Senadheera, S. N., Aldrich, J. V., and McLaughlin, J. P. (2013) The macrocyclic tetrapeptide [D-Trp]CJ-15,208 produces short-acting κ opioid receptor antagonism in the CNS after oral administration. Br J Pharmacol 169, 426-436]. Following determination of the individual initial preference as described above, the mice are subsequently place-conditioned immediately following administration of cocaine (10 mg·kg⁻¹, s.c.) and confined to a randomly assigned outer compartment starting on day 2. Place-conditioning in the opposite outer compartment is performed daily with vehicle (0.9% saline, 0.3 mL per 30 g body weight, s.c.) 4 h after the cocaine conditioning. This place-conditioning cycle is repeated once each day on days 3-5, and on day 6 the animals are tested for a final place preference.

Preference tests are performed twice weekly for 30 min until extinction is established. Extinction is defined as a statistically significant decrease in the time spent in the drug-paired compartment during the extinction trial as compared with the post-conditioning response after the initial 4 days of conditioning [Szumlinski, K. K., Price, K. L., Frys, K. A., and Middaugh L. D. (2002) Unconditioned and conditioned factors contribute to the ‘reinstatement’ of cocaine place conditioning following extinction in C57Bl/6 mice Behav Brain Res 136, 151-160; Brabant, C., Quertemont, E., and Tirelli, E. (2005) Influence of the dose and number of drug-context pairings on the magnitude and the long-lasting retention of cocaine-induced condition place preference in C57Bl/6 mice Psychopharmacology (Berl) 180, 33-40]. Generally, CCP responses show extinction around 3-8 weeks for the C57Bl/6 strain of mice.

Following the demonstration of extinction, reinstatement of CPP can be examined after either exposure to two days of forced swim stress or an additional cycle of drug place-conditioning as described above. Briefly, half the tested mice are pretreated with either vehicle or a compound of the invention daily 20 min prior to exposure to each of the two days of forced swimming [McLaughlin, J. P., Marton-Popovici, M., and Chavkin, C. (2003) Kappa opioid receptor antagonism and prodynorphin gene distribution block stress-induced behavioral response J Neurosci 23, 5674-5683; Carey, A. N., Borozny, K., Aldrich, J. V., and McLaughlin, J. P., (2007) Reinstatement of cocaine place-conditioning prevented by the peptide kappa-opioid receptor antagonist, Arodyn Eur J Pharmacol 569, 84-89]. Additional mice are also administered vehicle or the compound of the invention for two days, and 20 min after the final administration of vehicle or the compound of the invention an additional session of cocaine place conditioning is performed. On the day following the completion of stress exposure or cocaine place-conditioning, mice are tested for place preference (as described above). Reinstatement of cocaine CPP can also be examined for mice pretreated with either vehicle or a compound of the invention administered i.c.v. or peripherally (e.g. orally).

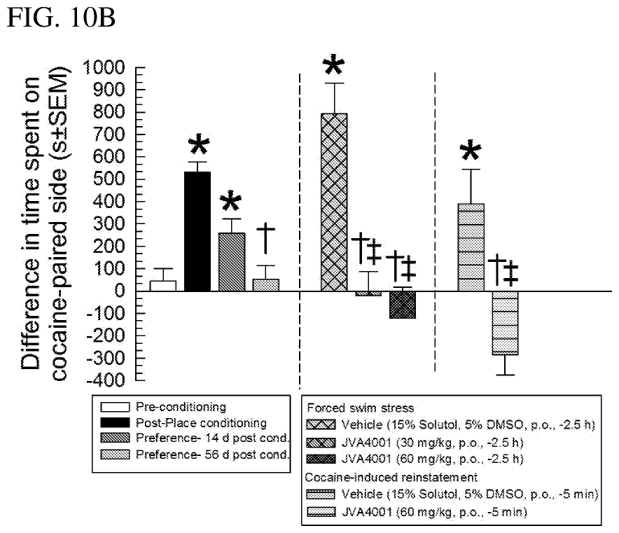

The macrocyclic tetrapeptide cyclo[D-Phe-Pro-Sar-Phe] was evaluated for its ability to prevent stress-induced reinstatement of cocaine conditioned place preference (CPP, FIGS. 10A-B). When administered at a dose (0.01 nmol i.c.v., −1.5 h) producing KOR antagonism, cyclo[D-Phe-Pro-Sar-Phe] prevented stress-induced reinstatement of cocaine CPP (FIG. 10A, center panel, blue bar). Of interest, a short (5 min) pretreatment with this dose also prevented cocaine-induced reinstatement (FIG. 10A, central panel, aqua bar). Notably, pretreatment with cyclo[D-Phe-Pro-Sar-Phe] alone under these conditions did not induce reinstatement in the mice tested (FIG. 10A, right panel, blue bar, n=8). When administered at oral doses (30 mg/kg and 60 mg/kg p.o., −2.5 h) producing KOR antagonism, cyclo[D-Phe-Pro-Sar-Phe] prevented stress-induced reinstatement of cocaine CPP (FIG. 10B).

### Morphine:

Mice were place conditioned 4 days with morphine (10 mg/kg, i.p.) as described above. Following 4 days of morphine place-conditioning, mice demonstrated a significant preference for the morphine-paired chamber (p<0.0001; FIG. 8A, black bar). Extinction of this preference was observed after repeated preference testing 8 weeks after conditioning (p<0.0001 vs. post-conditioning response; FIG. 8A, grey bar). Mice were then pretreated once daily for two days with vehicle (50% DMSO in 0.9% saline, i.c.v.) or cyclo[D-Phe-Pro-Sar-Phe] (0.01 or 0.03 nmol i.c.v.) and exposed to forced swim stress or an additional cycle of morphine place conditioning. Mice pretreated with vehicle displayed significant reinstatement of morphine CPP after exposure to two days of forced swimming or morphine place conditioning (p<0.0001; FIG. 8A). When pretreated with a dose and duration producing KOR antagonism (0.01 nmol i.c.v., −2.5 h), cyclo[D-Phe-Pro-Sar-Phe] prevented stress-induced reinstatement of morphine CPP (p=0.04). In contrast, when pretreated for 5 min when it exhibits agonist activity in the antinociception assay, cyclo[D-Phe-Pro-Sar-Phe] prevented morphine-induced reinstatement of morphine-CPP at a dose of 0.03 nmol (p=0.05), much as did the KOR-selective agonist U50,488 after treatment with a higher dose (100 nmol, i.c.v.; p=0.002; FIG. 8A). The cyclo[D-Phe-Pro-Sar-Phe]-induced prevention of morphine-induced reinstatement of morphine-CPP was dose-dependent, as pretreatment with a lower dose (0.01 nmol, i.c.v.) did not have significant effects (p=0.62). Notably, treatment with cyclo[D-Phe-Pro-Sar-Phe] alone at this dose did not directly reinstate extinguished CPP (rightmost bar, FIG. 8A), discounting any confounding effect of the cyclic tetrapeptide in this testing.

An additional set of mice significantly place conditioned 4 days with morphine subsequently demonstrated extinction (p<0.0001; FIG. 8B), and were then treated in week 10 with peripheral (i.p.) doses of vehicle, cyclo[D-Phe-Pro-Sar-Phe] or nor-BNI (10 mg/kg) prior to forced swimming. Mice pretreated with vehicle demonstrated significant reinstatement of morphine CPP after exposure to forced swimming (p<0.0001; FIG. 8B). Intraperitoneal pretreatment with nor-BNI (once 24 h prior to the start of forced swimming) or cyclo[D-Phe-Pro-Sar-Phe] (twice, 2.5 h prior to forced swimming daily) prevented stress-induced reinstatement (P=0.03 and 0.02, respectively; FIG. 8B, rightmost bars).

### Ethanol:

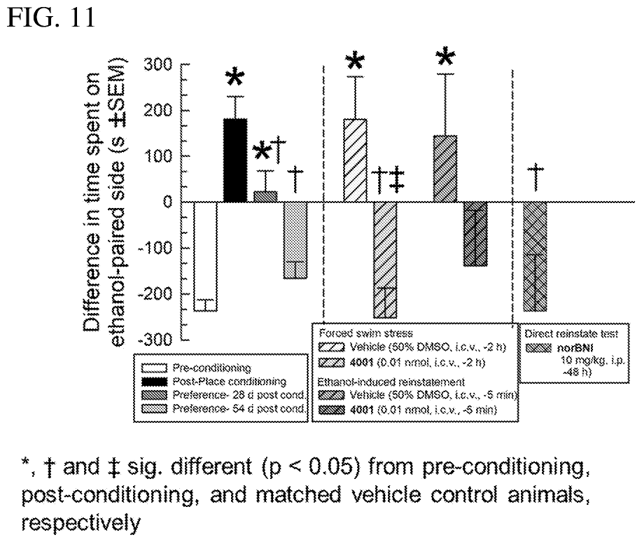

Similarly mice were place-conditioned using ethanol in a conditioned place-preference assay [Sperling, R. E., Gomes, S. M., Sypek, E. I., Carey, A. N. and McLaughlin, J. P., 2010. Endogenous kappa-opioid mediation of stress-induced potentiation of ethanol-conditioned place preference and self-administration. Psychopharmacology, 210, 199-209] with a biased conditioning paradigm (see McLaughlin, J. P., Ganno M. L., Eans, S. O., Mizrachi, E. and Paris, J. P., 2014. HIV-1 Tat protein exposure potentiates ethanol reward and reinstates extinguished ethanol-conditioned place preference. Current HIV Research, 12(6), 415-423). Following 4 days of ethanol conditioning mice subsequently exhibited extinction after 7-8 weeks (FIG. 11). Mice were then pretreated once daily for two days with vehicle (50% DMSO in 0.9% saline, i.c.v.) or cyclo[D-Phe-Pro-Sar-Phe] (0.01 nmol, i.c.v.) and exposed to two days of forced swim stress or an additional cycle of ethanol place conditioning as described above. Mice pretreated with vehicle displayed significant reinstatement of ethanol CPP after exposure to either forced swimming or an additional cycle of ethanol place conditioning. cyclo[D-Phe-Pro-Sar-Phe] prevented stress-induced reinstatement of ethanol CPP (FIG. 11) when administered at a dose and duration (0.01 nmol i.c.v., −2 h) producing KOR antagonism.

### Example 8: Rotarod Assay

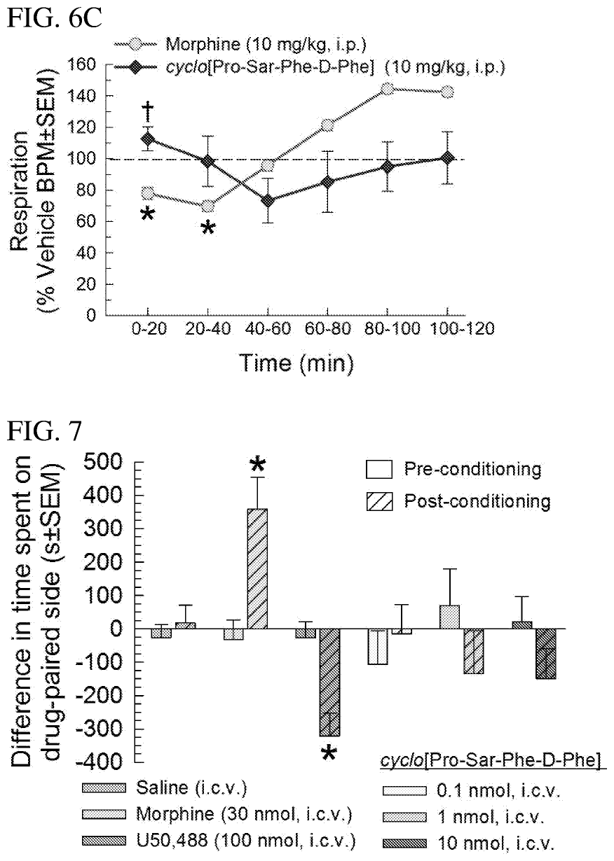

Both possible sedative and hyperlocomotor effects can be assessed by rotarod performance, as modified from previous protocols (see, Paris, J. J.; Reilley, K. J.; McLaughlin, J. P. J. Addiction Res Ther 2011, S4; and Aldrich, J V et al. 2013, J Nat Prod 76, 433-438; each of which are herein incorporated by reference in its entirety). Mice were first habituated to the rotarod over seven trials, with the last trial serving as the baseline response. Mice so habituated to the rotarod were then administered (i.p.) either saline, vehicle (10% Solutol in saline), U50,488 (10 mg/kg), or cyclo[D-Phe-Pro-Sar-Phe] (10 mg/kg) 15 min prior to assessment in accelerated speed trials (180 s max latency at 0-20 rpm) performed every 10 min over a 60 min period. Mice were thus tested a total of 14 trials (seven habituation trials prior to treatment+seven drug trials). Decreased latencies to fall in the rotarod test indicate impaired motor performance/sedation. Data are expressed as the percent change from baseline performance. A two-way ANOVA with Tukey HSD post hoc test can be used to analyze the data accordingly.

Whereas the KOR agonist (U50,488) at this dose significantly impaired coordinated locomotor activity in this assay compared to saline (p<0.0001), consistent with its sedative activity, cyclo[D-Phe-Pro-Sar-Phe] did not differ in effect from vehicle-treated animals (FIG. 6A).

### Example 9: Respiration and Ambulation

Respiration rates (in breaths per minute) and animal locomotive activity (as ambulations) were assessed using the Oxymax/CLAMS system (Columbus Instruments, Columbus, Ohio) as described previously [Armishaw, C. J., Banerjee, J., Ganno, M. L., Reilley, K. J., Eans, S. O., Mizrachi, E., Gyanda, R., Hoot, M. R., Houghten, R. A. and McLaughlin, J. P., 2013. Discovery of novel antinociceptive α-conotoxin analogues from the direct in vivo screening of a synthetic mixture-based combinatorial library. ACS Combinatorial Science, 15(3), 153-161; Hoot, M. R., Sypek, E. I., Reilley, K. J., Carey, A. N., Bidlack, J. M. and McLaughlin, J. P., 2013. Inhibition of Gβγ-subunit signaling potentiates morphine-induced antinociception but not respiratory depression, constipation, locomotion, and reward. Behavioural Pharmacology, 24(2), 144-152]. Mice were habituated to their individual sealed housing chambers for 60 min before testing. Mice were administered cyclo[D-Phe-Pro-Sar-Phe], vehicle, or morphine, as indicated, and five min later confined to the CLAMS testing cages. Pressure monitoring within the sealed chambers measured frequency of respiration. Infrared beams located in the floor measured locomotion as number of beam breaks. Respiration and locomotive data was averaged over 20 min periods for 120 min post-injection of the test compound.

The effect of cyclo[D-Phe-Pro-Sar-Phe] (JVA4001) or the MOR-preferring agonist morphine on spontaneous locomotor activity and respiration rate were assessed over 2 h after administration of a 10 mg/kg (i.p.) dose. Treatment with morphine produced a significant, time-dependent increase in ambulation (p<0.0001, FIG. 6B). In contrast, the cyclic tetrapeptide induced significantly less ambulation compared to morphine at later time points (40-120 min; p<0.05), which was not significantly elevated over the response of vehicle-treated animals (FIG. 6B). Treatment also had a generally significant effect on respiration over time (p=0.0002; FIG. 6C). However, whereas morphine significantly reduced respiration for the first 40 min compared to vehicle (p<0.005), cyclo[D-Phe-Pro-Sar-Phe] was without significant effect at any time point tested.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value. Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (1), or a solvate or hydrate thereof; 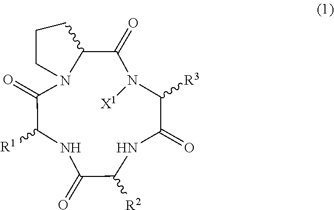
wherein
each R¹, R², and R³ is independently hydrogen or an amino acid side chain; and
X¹ is substituted or unsubstituted alkyl.

2. The compound of claim 1, wherein R¹ is a phenylalanine side chain.

3. The compound of claim 1, wherein R² is a phenylalanine side chain.

4. The compound of claim 1, wherein R³ is hydrogen.

5. The compound of claim 1, wherein X¹ is a C₁₋₆ alkyl.

6. The compound of claim 5, wherein X¹ is methyl.

7. The compound of claim 1 of Formula (1-aI), or a solvate or hydrate thereof; 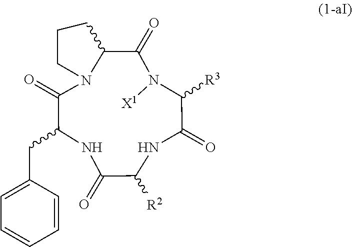
wherein
each R² and R³ is independently hydrogen or an amino acid side chain; and
X¹ is substituted or unsubstituted alkyl.

8. The compound of claim 7, wherein R² is a tryptophan side chain.

9. The compound of claim 7, wherein R² is not hydrogen.

10-12. (canceled)

13. The compound of claim 1 of Formula (1-aII), or a solvate or hydrate thereof; 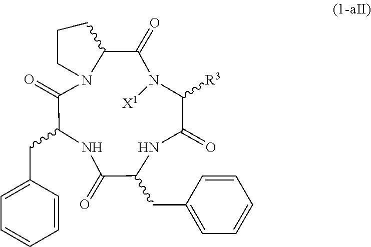
wherein
R³ is hydrogen or an amino acid side chain; and
X¹ is substituted or unsubstituted alkyl.

14. The compound of claim 1 of Formula (1-aIII), or a solvate or hydrate thereof; 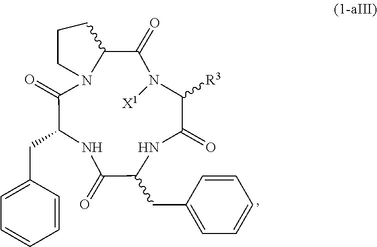
wherein
R³ is hydrogen or an amino acid side chain; and
X¹ is substituted or unsubstituted alkyl.

15. The compound of claim 1 of Formula (1-aIV), or a solvate or hydrate thereof; 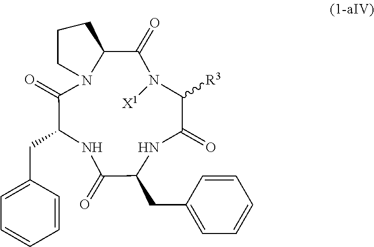
wherein
R³ is hydrogen or an amino acid side chain; and
X¹ is substituted or unsubstituted alkyl.

16-18. (canceled)

19. The compound of claim 1, wherein the compound is: 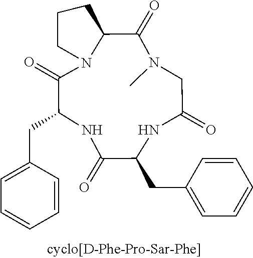 [SMILES: CN1CC(=O)N[C@@H](Cc2ccccc2)C(=O)N[C@H](Cc2ccccc2)C(=O)N2CCC[C@H]2C1=O]
or a solvate or hydrate thereof.

20. The compound of claim 1, wherein the compound is an opioid receptor agonist.

21-26. (canceled)

27. A pharmaceutical composition comprising the compound of claim 1 and a pharmaceutically acceptable adjuvant, carrier, or excipient.

28-33. (canceled)

34. A method of reducing or preventing the activation of an opioid receptor, the method comprising contacting the opioid receptor with an effective amount of the compound of claim 1.

35. A method of activating an opioid receptor, the method comprising contacting the opioid receptor with an effective amount of the compound of claim 1.

36-40. (canceled)

41. A method of reducing or preventing nociception, the method comprising administering to a subject in need thereof an effective amount of the compound of claim 1.

42. A method of treating a subject with a disease, disorder, or symptoms thereof, the method comprising administering to the subject an effective amount of a compound of claim 1.

43-58. (canceled)

59. A method of treating a subject suffering from a painful condition or symptoms thereof, the method comprising administering to the subject an effective amount of a compound of claim 1.

60-69. (canceled)
